SEQUENCE LISTING (36 sequences)

SEQ ID NO: 1 - protein, 6 aa, Artificial Sequence
  Polypeptide Linker
GGSGGS

SEQ ID NO: 2 - protein, 6 aa, Artificial Sequence
  Polypeptide Linker
GSSGSS

SEQ ID NO: 3 - protein, 15 aa, Artificial Sequence
  Polypeptide Linker
GGGGSGGGGSGGGGS

SEQ ID NO: 4 - protein, 8 aa, Artificial Sequence
  Polypeptide Linker
GGGGGGGG

SEQ ID NO: 5 - protein, 6 aa, Artificial Sequence
  Polypeptide Linker
GGGGGG

SEQ ID NO: 6 - protein, 15 aa, Artificial Sequence
  Polypeptide Linker
EAAAKEAAAKEAAAK

SEQ ID NO: 7 - protein, 5 aa, Artificial Sequence
  Polypeptide Linker
EAAAK

SEQ ID NO: 8 - protein, 10 aa, Artificial Sequence
  Polypeptide Linker
EAAAKEAAAK

SEQ ID NO: 9 - protein, 46 aa, Artificial Sequence
  Polypeptide Linker
AEAAAKEAAAKEAAAKEAAAKALEAEAAAKEAAAKEAAAKEAAAKA

SEQ ID NO: 10 - protein, 5 aa, Artificial Sequence
  Polypeptide Linker
GGGGS

SEQ ID NO: 11 - protein, 5 aa, Artificial Sequence
  Polypeptide Linker
PAPAP

SEQ ID NO: 12 - protein, 12 aa, Artificial Sequence
  Polypeptide Linker
AEAAAKEAAAKA

SEQ ID NO: 13 - protein, 10 aa, Artificial Sequence
  Polypeptide Linker
GGGGSGGGGS

SEQ ID NO: 14 - protein, 20 aa, Artificial Sequence
  Polypeptide Linker
GGGGSGGGGSGGGGSGGGGS

SEQ ID NO: 15 - protein, 10 aa, Artificial Sequence
  Polypeptide Linker
APAPAPAPAP

SEQ ID NO: 16 - protein, 12 aa, Artificial Sequence
  Polypeptide Linker
APAPAPAPAPAP

SEQ ID NO: 17 - protein, 14 aa, Artificial Sequence
  Polypeptide Linker
APAPAPAPAPAPAP

SEQ ID NO: 18 - protein, 16 aa, Artificial Sequence
  Polypeptide Linker
APAPAPAPAPAPAPAP

SEQ ID NO: 19 - protein, 18 aa, Artificial Sequence
  Polypeptide Linker
APAPAPAPAPAPAPAPAP

SEQ ID NO: 20 - protein, 20 aa, Artificial Sequence
  Polypeptide Linker
APAPAPAPAPAPAPAPAPAP

SEQ ID NO: 21 - protein, 22 aa, Artificial Sequence
  Polypeptide Linker
APAPAPAPAPAPAPAPAPAPAP

SEQ ID NO: 22 - protein, 24 aa, Artificial Sequence
  Polypeptide Linker
APAPAPAPAPAPAPAPAPAPAPAP

SEQ ID NO: 23 - protein, 26 aa, Artificial Sequence
  Polypeptide Linker
APAPAPAPAPAPAPAPAPAPAPAPAP

SEQ ID NO: 24 - protein, 28 aa, Artificial Sequence
  Polypeptide Linker
APAPAPAPAPAPAPAPAPAPAPAPAPAP

SEQ ID NO: 25 - protein, 30 aa, Artificial Sequence
  Polypeptide Linker
APAPAPAPAPAPAPAPAPAPAPAPAPAPAP

SEQ ID NO: 26 - protein, 32 aa, Artificial Sequence
  Polypeptide Linker
APAPAPAPAPAPAPAPAPAPAPAPAPAPAPAP

SEQ ID NO: 27 - protein, 34 aa, Artificial Sequence
  Polypeptide Linker
APAPAPAPAPAPAPAPAPAPAPAPAPAPAPAPAP

SEQ ID NO: 28 - protein, 17 aa, Artificial Sequence
  Polypeptide Linker
VSQTSKLTRAETVFPDV

SEQ ID NO: 29 - protein, 6 aa, Artificial Sequence
  Polypeptide Linker
PLGLWA

SEQ ID NO: 30 - protein, 6 aa, Artificial Sequence
  Polypeptide Linker
RVLAEA

SEQ ID NO: 31 - protein, 10 aa, Artificial Sequence
  Polypeptide Linker
EDVVCCSMSY

SEQ ID NO: 32 - protein, 8 aa, Artificial Sequence
  Polypeptide Linker
GGIEGRGS

SEQ ID NO: 33 - protein, 10 aa, Artificial Sequence
  Polypeptide Linker
TRHRQPRGWE

SEQ ID NO: 34 - protein, 10 aa, Artificial Sequence
  Polypeptide Linker
AGNRVRRSVG

SEQ ID NO: 35 - protein, 9 aa, Artificial Sequence
  Polypeptide Linker
RRRRRRRRR

SEQ ID NO: 36 - protein, 4 aa, Artificial Sequence
  Polypeptide Linker
GFLG

(19) United States
(12) Patent Application Publication
(10) Pub. No.: US 2025/0320552 A1
(43) Pub. Date: Oct. 16, 2025

(54) DETECTING METHYLCYTOSINE USING A MODIFIED BASE OPPOSITE TO THE METHYLCYTOSINE

(71) Applicant: ILLUMINA, INC., San Diego, CA (US)

(72) Inventors: Colin Brown, San Diego, CA (US); Sarah Shultzaberger, San Diego, CA (US); Eric Brustad, San Diego, CA (US)

(73) Assignee: ILLUMINA, INC., San Diego, CA (US)

(21) Appl. No.: 18/568,366

(22) PCT Filed: Jun. 30, 2022
(86) PCT No.: PCT/US2022/035786

§ 371 Date: Dec. 8, 2023

Related U.S. Application Data
(60) Provisional application No. 63/218,168, filed on Jul. 2, 2021.

(51) Int. Cl. C12Q 1/6876; C12Q 1/6804; C12Q 1/6825

(52) U.S. Cl. CPC C12Q 1/6876; C12Q 1/6804; C12Q 1/6825; C12Q 2600/154

(57) ABSTRACT

Examples provided herein are related to detecting methylcytosine using a modified base opposite to the methylcytosine. A methylcytosine in a first polynucleotide including a plurality of cytosines may be detected, using a method that includes hybridizing the first polynucleotide to a second polynucleotide. The second polynucleotide may include a modified base opposite to the methylcytosine. The methylcytosine may be detected using the modified base. For example, the modified base may include a fluorophore. The methylcytosine may be detected using fluorescence from the fluorophore responsive to excitation light.

### CROSS-REFERENCE TO RELATED APPLICATION

This application claims the benefit of U.S. Provisional Patent Application No. 63/218,168, filed on Jul. 2, 2021 and entitled “DETECTING METHYLCYTOSINE USING A MODIFIED BASE OPPOSITE TO THE METHYLCYTOSINE”, the entire contents of which are incorporated by reference herein.

### FIELD

This application relates to methods for detecting methylcytosine.

### SEQUENCE LISTING

The instant application contains a Sequence Listing which has been submitted electronically in ASCII format and is hereby incorporated by reference in its entirety. Said ASCII copy, created on Jun. 27, 2022, is named IP-2068-PCT_SL and is 8,793 bytes in size.

### BACKGROUND

Within living organisms, such as humans, selected cytosines (Cs) in the genome may become methylated. For example, S-adenosyl-L-methionine (SAM) is known to be a ubiquitous methyl donor for a variety of biological methylation reactions that are catalyzed by enzymes referred to as methyltransferases (MTases). The enzyme 5-MTase may add a methyl group to the 5-position of cytosine to form 5-methylcytosine (5mC) in a manner such as described in Deen et al., “Methyltransferase-directed labeling of biomolecules and its applications,” Angewandte Chemie International Edition 56: 5182-5200 (2017), the entire contents of which are incorporated by reference herein. Other enzyme(s) may oxidize the cytosine's methyl group to form the 5mC derivative 5-hydroxymethyl cytosine (5hmC), and may oxidize the 5hmC further to form the 5mC derivatives 5-formyl cytosine (5fC) and 5-carboxy cytosine (5caC).

5mC and 5hmC may be referred to as epigenetic markers, and it can be desirable to detect them in a genomic sequence. For example, 5mC is proposed to have diverse roles in regulation of gene expression, parental imprinting, and molecular etiology of human diseases including cancer. Hundreds of methylation biomarkers have been discovered for cancer and other diseases, and methylation signatures in circulating cell-free DNA (cfDNA) have shown promise as a basis for liquid biopsy assays for diagnoses, treatment selection, and disease monitoring.

Two broad categories of approaches have been developed to measure DNA methylation. Enrichment strategies select methylated DNA fragments using a 5mC-specific antibody, methylation-sensitive restriction enzymes, or methylation-induced changes in DNA duplex stability. The methylated DNA fragments then can be measured in relation to a non-enriched sample by qPCR or other standard nucleic acid quantitation strategies. Methylation assays based on chemical transformation begin by treating the sample with a chemical or enzymatic reagent that creates a difference in base pairing between methylated and non-methylated cytosine residues. The current golden standard method for detecting 5mC and 5hmC is bisulfite sequencing, which converts any unmethylated C in the sequence to uracil (U), but does not convert 5mC or 5hmC to the corresponding uracil derivatives. When the sequence is amplified using polymerase chain reaction (PCR), the uracil is amplified as thymidine (T), and as such the unmethylated C is sequenced as T. In comparison, the 5mC and 5hmC are amplified as C, and as such are sequenced as C. Thus, any Cs in the sequence may be identified as corresponding to 5mC or 5hmC because they had not been converted to U. Such a scheme may be referred to as a “three-base” sequencing scheme because any unmethylated C is converted to T. However, this type of scheme reduces sequence complexity and may lead to reduced sequencing quality, lower mapping rates, and relatively uneven coverage of the sequence.

Despite the importance of DNA methylation in the etiology of many human diseases, and the identification of hundreds of methylation biomarkers for cancer and other disorders, only a small number of methylation-based diagnostic assays have been adopted for use in the clinic. A major reason for this discrepancy is the relative difficulty of measuring cytosine methylation as compared to SNPs and other DNA sequence changes. Cytosine methylation is a relatively minor chemical change in the structure of the nucleobase, and on its own does not change the pattern of hydrogen bond donors and acceptors that govern specific base pairing.

### SUMMARY

Examples provided herein are related to detecting methylcytosine using a modified base opposite to the methylcytosine. Compositions and methods for performing such detection are disclosed.

Some examples herein provide a method for detecting a methylcytosine in a first polynucleotide including a plurality of cytosines. The method may include hybridizing the first polynucleotide to a second polynucleotide. The second polynucleotide includes a modified base opposite to the methylcytosine. The method may include detecting the methylcytosine using the modified base.

In some examples, the modified base includes a fluorophore. In some examples, the methylcytosine is detected using fluorescence from the fluorophore responsive to excitation light. In some examples, the fluorescence is induced using a first protein. In some examples, the first protein couples to the methylcytosine. In some examples, the coupling of the first protein to the methylcytosine dissociates the methylcytosine from the modified base while the first polynucleotide remains hybridized to the second polynucleotide. In some examples, responsive to the dissociation of the methylcytosine from the modified base, the fluorophore fluoresces at a first intensity and a first wavelength. In some examples, responsive to the dissociation of the methylcytosine from the modified base while the first polynucleotide remains hybridized to the second polynucleotide, the fluorophore fluoresces at a second intensity and a second wavelength. In some examples, the second intensity is different than the first intensity. Additionally, or alternatively, in some examples, the second wavelength is different than the first wavelength.

Additionally, or alternatively, in some examples, the modified base includes a solvatochromatic nucleoside.

Additionally, or alternatively, in some examples, the modified base includes a modified guanine or a modified adenine.

Additionally, or alternatively, in some examples, the modified base includes a first target. In some examples, the method further includes coupling the methylcytosine to a first protein. The first protein may be coupled to a second protein, and the second protein selectively binds to the first target when the first protein couples to the methylcytosine. In some examples, a fluorophore is coupled to the second protein. Alternatively, in some examples, the second protein includes a second target. The fluorophore may be coupled to a third protein that selectively binds to the second target. In some examples, the second target includes an epitope, and wherein the third protein includes an antibody. Additionally, or alternatively, in some examples, the first and second proteins include different parts of a fusion protein. Additionally, or alternatively, in some examples, the first protein is coupled to the second protein via a second linker. Additionally, or alternatively, in some examples, the second protein includes a SNAP protein and the first target includes an O-benzylguanine. Alternatively, in some examples, the second protein includes a CLIP protein and the first target includes an O-benzylcytosine. Alternatively, in some examples, the second protein includes SpyTag and the first target includes SpyCatcher, or the second protein includes SpyCatcher and the first target includes SpyTag. Alternatively, in some examples, the second protein includes biotin and the first target includes streptavidin, or the second protein includes streptavidin and the first target includes biotin. Alternatively, in some examples, the second protein includes NTA and wherein the first target includes His-Tag, or the second protein includes His-Tag and the first target includes NTA.

Additionally, or alternatively, in some examples the first protein is coupled to a first half of a split fluorophore; the second protein is coupled to a second half of a split fluorophore; and the first half of the split fluorophore becomes coupled to the second half of the split fluorophore when the first protein becomes coupled to the methylcytosine to induce fluorescence.

Additionally, or alternatively, in some examples, the first protein includes a methyl binding protein (MBP).

Additionally, or alternatively, in some examples, the first protein includes a SET and Ring finger Associated (SRA) domain.

Additionally, or alternatively, in some examples, the modified base is coupled to a fluorophore after the first polynucleotide is hybridized to the second polynucleotide.

Additionally, or alternatively, in some examples, the second polynucleotide is directly coupled to a substrate. Additionally, or alternatively, in some examples, the second polynucleotide is hybridized to a third polynucleotide that is directly coupled to a substrate. In some examples, the substrate is coupled to an oligonucleotide including a code identifying the first polynucleotide. In some examples, the oligonucleotide is coupled to the substrate separately from the second polynucleotide. In some examples, the oligonucleotide couples the second polynucleotide to the substrate. Additionally, or alternatively, in some examples, the substrate includes a bead. Additionally, or alternatively, in some examples, detecting the methylcytosine using the modified base includes identifying the first polynucleotide using the code.

Some examples herein provide a composition. The composition may include a first polynucleotide hybridized to a second polynucleotide. The first polynucleotide may include a methylcytosine and a plurality of cytosines. The second polynucleotide may include a modified base opposite to the methylcytosine. The modified base may include a detectable moiety.

In some examples, the detectable moiety includes a fluorophore. In some examples, the methylcytosine is detectable using fluorescence from the fluorophore responsive to excitation light. Some examples further include a first protein inducing the fluorescence. In some examples, the first protein is coupled to the methylcytosine. In some examples, the coupling between the first protein and the methylcytosine dissociates the methylcytosine from the modified base while the first polynucleotide remains hybridized to the second polynucleotide. In some examples, responsive to association of the methylcytosine to the modified base, the fluorophore fluoresces at a first intensity and a first wavelength. In some examples, responsive to the dissociation of the methylcytosine from the modified base while the first polynucleotide remains hybridized to the second polynucleotide, the fluorophore fluoresces at a second intensity and a second wavelength. In some examples, the second intensity is different than the first intensity. Additionally, or alternatively, in some examples, the second wavelength is different than the first wavelength. Additionally, or alternatively, in some examples, the modified base includes a solvatochromatic nucleoside. Additionally, or alternatively, in some examples, the modified base includes a modified guanine or a modified adenine.

Additionally, or alternatively, in some examples, the modified base includes a first target. In some examples, the methylcytosine is coupled to a first protein. The first protein may be coupled to a second protein, and the second protein selectively binds to the first target when the first protein couples to the methylcytosine. Additionally, or alternatively, in some examples, the first protein is coupled to a first half of a split fluorophore, and the second protein is coupled to a second half of a split fluorophore. The first half of the split fluorophore may become coupled to the second half of the split fluorophore when the first protein becomes coupled to the methylcytosine to induce fluorescence.

Additionally, or alternatively, in some examples, the fluorophore is coupled to the second protein. In some examples, the second protein includes a second target, and the fluorophore is coupled to a third protein that selectively binds to the second target. In some examples, the second target includes an epitope, and the third protein includes an antibody. Additionally, or alternatively, in some examples, the first and second proteins include different parts of a fusion protein. Additionally, or alternatively, in some examples, the first protein is coupled to the second protein via a second linker. Additionally, or alternatively, in some examples, the second protein includes a SNAP protein and the first target includes an O-benzylguanine. Additionally, or alternatively, in some examples, the second protein includes a CLIP protein and the first target includes an O-benzylcytosine. Additionally, or alternatively, in some examples, the second protein includes SpyTag and the first target includes SpyCatcher, or the second protein includes SpyCatcher and the first target includes SpyTag. Additionally, or alternatively, in some examples, the second protein includes biotin and the first target includes streptavidin, or the second protein includes streptavidin and the first target includes biotin. Additionally, or alternatively, in some examples, the second protein includes NTA and the first target includes His-Tag, or the second protein includes His-Tag and the first target includes NTA.

Additionally, or alternatively, in some examples, the first protein includes a methyl binding protein (MBP).

Additionally, or alternatively, in some examples, the first protein includes a SET and Ring finger Associated (SRA) domain.

Additionally, or alternatively, in some examples, the modified base is coupled to the fluorophore after the first polynucleotide is hybridized to the second polynucleotide.

Additionally, or alternatively, in some examples, the second polynucleotide is directly coupled to a substrate. Alternatively, in some examples, the second polynucleotide is hybridized to a third polynucleotide that is directly coupled to a substrate. Additionally, or alternatively, in some examples, the substrate is coupled to an oligonucleotide including a code identifying the first polynucleotide. In some examples, the oligonucleotide is coupled to the substrate separately from the second polynucleotide. Alternatively, in some examples, the oligonucleotide couples the second polynucleotide to the substrate. Additionally, or alternatively, in some examples, the substrate includes a bead. Additionally, or alternatively, in some examples, detecting the methylcytosine using the modified base includes identifying the first polynucleotide using the code.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1C schematically illustrate example compositions and operations in a process flow for detecting methylcytosine using a modified base opposite to the methylcytosine.

FIGS. 2A-2C schematically illustrate example compositions and operations in a process flow for detecting methylcytosine using a modified base including a fluorophore.

FIGS. 3A-3H schematically illustrate additional example compositions and operations in a process flow for detecting methylcytosine using a modified base including a fluorophore.

FIGS. 4A-4D schematically illustrate example compositions and operations in a process flow for detecting methylcytosine using a modified base including a target to which a fluorophore may be coupled.

FIGS. 5A-5H schematically illustrate additional example compositions and operations in a process flow for detecting methylcytosine using a modified base including a target to which a fluorophore may be coupled.

FIGS. 6A-6D schematically illustrate additional example compositions and operations in a process for detecting methylcytosine using a modified base opposite to the methylcytosine.

FIGS. 7A-7B schematically illustrate example compositions and operations in a process flow for detecting methylcytosine using a modified base opposite to the methylcytosine.

FIG. 8 illustrates a flow of operations in an example method for detecting methylcytosine using a modified base opposite to the methylcytosine.

### DETAILED DESCRIPTION

Examples provided herein are related to detecting methylcytosine using a modified base opposite to the methylcytosine. Compositions and methods for performing such detection are disclosed.

Provided herein is site-specific, direct detection of cytosine methylation in which a modified base opposite to the methylcytosine (e.g., a modified base to which the methylcytosine hybridizes) generates a signal indicative of the methylcytosine. In a manner such as described in greater detail below, the signal may be generated using a 5mC-binding protein domain that binds to the methylcytosine. In some examples, the modified base may include a fluorophore, and the 5mC-binding protein domain may dissociate (e.g., dehybridize) the methylcytosine from the modified base to which it is opposite. Responsive to such dissociation, the intensity, the wavelength, or both the intensity and the wavelength of the fluorophore's fluorescence may detectably change, and such change may be correlated to the presence of methylcytosine to which the 5mC-binding protein domain bound. In other examples, the modified base may include a target, and the 5mC-binding protein domain may be coupled to a target partner (such as a protein) that selectively binds to that target. A fluorophore may be coupled to the target partner or the target, or both, and fluorescence from the fluorophore may be correlated to the presence of methylcytosine that was bound by the 5mC-binding protein domain.

First, some terms used herein will be briefly explained. Then, some example compositions and example methods will be described for detecting methylcytosine using a modified base opposite to the methylcytosine.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes disassociate from one another. When the first and second polynucleotide are hybridized to one another, pairs of bases may be “opposite” to each other, and the bases of that pair may be said to “associate” with each other. When bases of a given pair are complementary to each other, those bases also may be said to “hybridize” to one another. On the other hand, when one base of a given pair is pulled away from the other base of that pair, the bases may be said to “disassociate” from each other.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue (also referred to as a modified base) which is a type of nucleotide that includes a modified nucleobase, sugar and/or phosphate moiety compared to naturally occurring nucleotides. Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. Other modified bases may include targets and/or fluorophores in a manner such as described elsewhere herein. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Example polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” refers to a polynucleotide to which nucleotides may be added via a free 3′ OH group. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer. A primer may be coupled to a substrate.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible. In some examples, a substrate is a bead or a flow cell.

In some examples, a substrate includes a patterned surface. A “patterned surface” refers to an arrangement of different regions in or on an exposed layer of a substrate. For example, one or more of the regions may be features where one or more capture primers are present. The features can be separated by interstitial regions where capture primers are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of wells (e.g., microwells or nanowells) on glass, silicon, plastic or other suitable material(s) with patterned, covalently-linked gel such as poly(N-(5-azidoacetamidylpentyl) acrylamide-co-acrylamide) (PAZAM). The process creates gel pads used for sequencing that may be stable over sequencing runs with a large number of cycles. The covalent linking of the polymer to the wells may be helpful for maintaining the gel in the structured features throughout the lifetime of the structured substrate during a variety of uses. However in many examples, the gel need not be covalently linked to the wells. For example, in some conditions silane free acrylamide (SFA) which is not covalently attached to any part of the structured substrate, may be used as the gel material.

In particular examples, a structured substrate may be made by patterning a suitable material with wells (e.g. microwells or nanowells), coating the patterned material with a gel material (e.g., PAZAM, SFA or chemically modified variants thereof, such as the azidolyzed version of SFA (azido-SFA)) and polishing the surface of the gel coated material, for example via chemical or mechanical polishing, thereby retaining gel in the wells but removing or inactivating substantially all of the gel from the interstitial regions on the surface of the structured substrate between the wells. Primers may be attached to gel material. A solution including a plurality of target polynucleotides (e.g., a fragmented human genome or portion thereof) may then be contacted with the polished substrate such that individual target polynucleotides will seed individual wells via interactions with primers attached to the gel material; however, the target polynucleotides will not occupy the interstitial regions due to absence or inactivity of the gel material. Amplification of the target polynucleotides may be confined to the wells because absence or inactivity of gel in the interstitial regions may inhibit outward migration of the growing cluster. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An example patterned substrate having these characteristics is the etched substrate used in connection with BEAD ARRAY technology (Illumina, Inc., San Diego, CA).

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, CA).

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Example polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “methylcytosine” or “mC” refers to cytosine in DNA (namely, 2′-deoxycytosine) that includes a methyl group (—CH₃ or -Me), or a derivative of methylcytosine. As used herein, a “derivative” of methylcytosine refers to methylcytosine having a methyl group or a derivatized methyl group. A nonlimiting example of a derivatized methyl group is an oxidized methyl group. A nonlimiting example of an oxidized methyl group is hydroxymethyl (—CH₂OH), in which case the mC derivative may be referred to as hydroxymethylcytosine or hmC. Another nonlimiting example of an oxidized methyl group is formyl group (—CHO) in which case the mC derivative may be referred to as formylcytosine or fC. Another nonlimiting example of an oxidized methyl group is carboxyl (—COOH), in which case the mC derivative may be referred to as carboxycytosine or caC. The methyl group may be located at the 5 position of the cytosine, in which case the mC may be referred to as 5mC. The oxidized methyl group may be located at the 5 position of the cytosine, in which case the hmC may be referred to as 5hmC, the fC may be referred to as 5fC, or the caC may be referred to as 5caC. Another nonlimiting example of a derivatized methyl group is a glucosylated methyl group. For example, the mC derivative may be glucosylated hmC. Glucosylated hmC may be produced by T4 beta-glucosyltransferase.

As used herein, the term “fluorophore” is intended to mean a molecule that emits light at a first wavelength responsive to excitation with light at a second wavelength that is different from the first wavelength. The light emitted by a fluorophore may be referred to as “fluorescence” and may be detected by suitable optical circuitry. Example fluorophores include dyes and solvatochromatic nucleosides.

By “solvatochromatic nucleoside” it is meant a modified base that includes a fluorescent nucleoside analog with context-dependent spectral properties. For example, a solvatochromatic nucleoside may fluoresce at a first wavelength and a first intensity when associated with a nucleoside to which the solvatochromatic nucleoside is opposite (e.g., when hybridized to a complementary nucleoside to which the solvatochromatic nucleoside is opposite), and may fluoresce at a second wavelength and a second intensity when across from an abasic site, where the second wavelength or the second intensity, or both, differs from the first wavelength or the first intensity. Illustratively, the solvatochromatic nucleoside may include a modified guanosine, and may fluoresce at a first wavelength and a first intensity when hybridized to cytosine or methylcytosine, and may fluoresce at a second wavelength and a second intensity when across from an abasic site, where the second wavelength or the second intensity, or both, differs from the first wavelength or the first intensity. A modified nucleotide incorporating such a modified guanosine may be referred to herein as a modified guanine. Alternatively, the solvatochromatic nucleoside may include a modified adenosine, and may fluoresce at a first wavelength and a first intensity when opposite to a cytosine or methylcytosine, and may fluoresce at a second wavelength and a second intensity when across from an abasic site, where the second wavelength or the second intensity, or both, differs from the first wavelength or the first intensity. A modified nucleotide incorporating such a modified adenosine may be referred to herein as a modified adenine.

Nonlimiting examples of solvatochromatic guanosines include: an ethynyl-modified 3-deaza-2′-deoxyguanosine, a 3-naphthylethynylated 3-deaza-2′deoxyguanosine, 3-(1-ethynylpyrenyl)-3-deaza-2′-deoxyguanosine, a 8-styryl-2′-deoxyguanosine, 8-azaguanine (8-AzaG), deoxythienoguanosine (d^{th}G), 1,6-disubstituted guanosine derivatives (such as 1,6-^{CN}G or 1,6-^{Ac}G), or 7-deazaguanine derivatives directly modified with an aryl group, such as listed below:

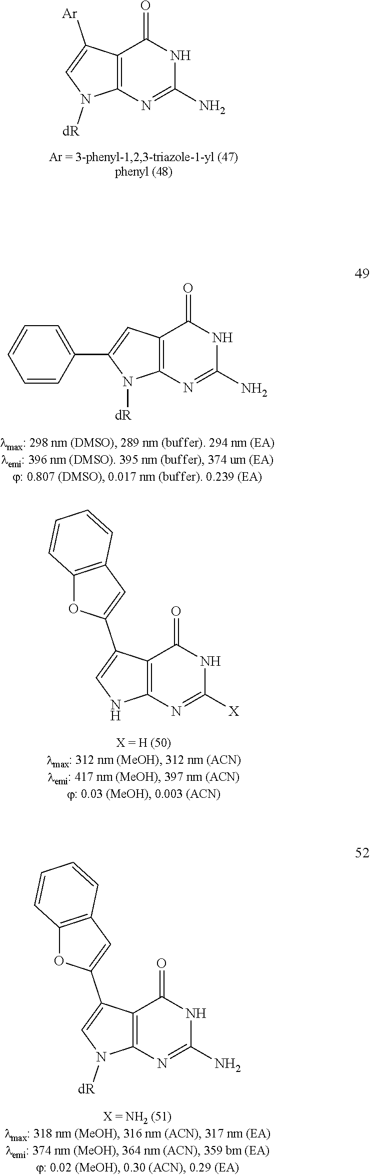

Nonlimiting examples of solvatochromatic adenines include a 8-styryl-2′-deoxyadenosine or a C2-substituted 8-aza-7-deaza-2′-deoxyadenosine. For further details regarding these and other example solvatochromatic nucleosides that may be used in the present compositions and methods, see the following references, the entire contents of each of which are incorporated by reference herein: Takeda et al., “Synthesis of ethynylpyrene-modified 3-deaza-2′-deoxyguanosines as environmentally sensitive fluorescent nucleosides: Target DNA-sequence detection via changes in the fluorescence wavelength,” Tetrahedron Letters 60 (12): 825-830 (2019); Saito et al., “An environmentally sensitive purine nucleoside that changes emission wavelength upon hybridization,” Chemical Communications 49(50): 5684-5686 (2013); Seio et al., “Solvent- and environment-dependence fluorescence of modified nucleobases,” Tetrahedron Letters 59: 1977-1985 (2018); Matsumoto et al., “Design and synthesis of highly solvatochromatic fluorescent 2′-deoxyguanosine and 2′-deoxyadenosine analogs,” Bioorganic & Medicinal Chemistry Letters 21(4): 1275-1278 (2011); Xu et al., “Fluorescent nucleobases as tools for studying DNA and RNA,” Nat. Chem. 9(11): 1043-1055 (2017); Sholokh et al., “Conquering 2-aminopurine's deficiencies: Highly emissive isomorphic guanosine surrogate faithfully monitors guanosine conformation and dynamics in DNA,” J. Am. Chem. Soc. 137(9): 3185-3188 (2015); Saito et al., “Synthesis of novel push-pull-type solvatochromatic 2′-deoxyguanosine derivatives with longer wavelength emission”.

As used herein, to “detect” fluorescence is intended to mean to receive light from a fluorophore, to generate an electrical signal based on the received light, and to determine, using the electrical signal, that light was received from the fluorophore. Fluorescence may be detected using any suitable optical detection circuitry, which may include an optical detector to generate an electrical signal based on the light received from the fluorophore, and electronic circuitry to determine, using the electrical signal, that light was received from the fluorophore. As one example, the optical detector may include an active-pixel sensor (APS) including an array of amplified photodetectors configured to generate an electrical signal based on light received by the photodetectors. APSs may be based on complementary metal oxide semiconductor (CMOS) technology known in the art. CMOS-based detectors may include field effect transistors (FETs), e.g., metal oxide semiconductor field effect transistors (MOSFETs). In particular examples, a CMOS imager having a single-photon avalanche diode (CMOS-SPAD) may be used, for example, to perform fluorescence lifetime imaging (FLIM). In other examples, the optical detector may include a photodiode, such as an avalanche photodiode, charge-coupled device (CCD), cryogenic photon detector, reverse-biased light emitting diode (LED), photoresistor, phototransistor, photovoltaic cell, photomultiplier tube (PMT), quantum dot photoconductor or photodiode, or the like. The optical detection circuitry further may include any suitable combination of hardware and software in operable communication with the optical detector so as to receive the electrical signal therefrom, and configured to detect the fluorescence based on such signal, e.g., based on the optical detector detecting light from the fluorophore. For example, the electronic circuitry may include a memory and a processor coupled to the memory. The memory may store instructions for causing the processor to receive the signal from the optical detector and to detect the fluorophore using such signal. For example, the instructions can cause the processor to determine, using the signal from the optical detector, that fluorescence is emitted within the field of view of the optical detector and to determine, using such determination, that a fluorophore is present.

To “measure” fluorescence is intended to mean to determine a relative or absolute amount of the fluorescence that is detected. For example, the amount of fluorescence may be measured relative to a baseline amount of fluorescence, or as an absolute amount of fluorescence. Illustratively, the amount of fluorescence from one or more fluorophores may be correlated to the amount of a modified base, in a polynucleotide, that is hybridized to methylcytosine. For example, the memory of the electronic circuitry described above may store instructions causing the processor to monitor the level of the electrical signal at one or more times, and to correlate such level(s) to an amount of methylcytosine.

As used herein, “methyl-binding protein” or “MBP” is intended to mean a protein or protein domain that specifically binds methylcytosine in dsDNA. Some MBPs may specifically bind multiple different methylcytosine derivatives, while some MBPs may specifically bind a particular methylcytosine derivatives. For example, the binding affinities of human MBD family proteins hMBD1-4 and hMeCP2 are described in Buchmuller et al., “Complete profiling of methyl-CpG-binding domains for combinations of cytosine modifications at CpG dinucleotides reveals differential read-out in normal and Rett-associated states,” Scientific Reports 10, Article number: 4053 (2020), the entire contents of which are incorporated by reference herein. As another example, the relative binding affinity of the SUVH5 SRA domain for hmC, meC, fC, and caC is disclosed in Rajakumara et al., “Mechanistic insights into the recognition of 5-methycytosine oxidation derivatives by the SUVH5 SRA domain,” Scientific reports 6, Article number: 20161 (2016), the entire contents of which are incorporated by reference herein. As another example, the relative binding affinity of UHRF2 for different methylcytosine derivatives is disclosed in Hashimoto et al., “The SRA domain of UHRF1 flips 5-methylcytosine out of the DNA helix,” Nature 455: 826-830 (2008); and Spruijt et al., “Dynamic readers for 5-(hydroxy)methylcytosine and its oxidized derivatives,” Cell 152(5): 1146-1159 (2013); the entire contents of each of which are incorporated by reference herein.

Although many methyl-binding proteins are known, the perhaps best-studied examples include a SET and RING-associated (SRA) domain. These domains can be expressed and purified independently of their parent proteins and utilize a “base-flipping” mechanism for methylcytosine recognition in which the methylated base is rotated out of the dsDNA duplex into a binding pocket on the protein. For further details regarding the structure and activity of the SRA domain and proteins that may include it, see the following references, the entire contents of each of which are incorporated by reference herein: Greiner et al., “Site-selective monitoring of the interaction of the SRA domain of UHRF1 with target DNA sequences labeled with 2-aminopurine,” Biochemistry 54: 6012-6020 (2015); Kilin et al., “Dynamics of methylated cytosine flipping by UHRF1,” JACS 139(6): 2520-2528 (2017); Zhou et al., “Structural basis for hydroxymethylcytosine recognition by the SRA domain of UHRF2,” Molecular Cell 54: 879-886 (2014); Hashimoto et al., “The SRA domain of UHRF1 flips 5-methylcytosine out of the DNA helix,” Nature 455: 826-830 (2008); and Avvakumov et al., “Structural basis for recognition of hemi-methylated DNA by the SRA domain of human UHRF1,” Nature 455(7214): 822-825 (2008). Other MBPs include MBD family proteins such as described in Buchmuller et al., “Complete profiling of methyl-CpG-binding domains for combinations of cytosine modifications at CpG dinucleotides reveals differential read-out in normal and Rett-associated states,” Scientific Reports 10, Article number: 4053 (2020), the entire contents of which are incorporated by reference herein. Other MBPs include Kaiso family proteins such as described in Filion et al., “A family of human zinc finger proteins that bind methylated DNA and repress transcription,” Mol. Cell Biol. 26(1): 169-181 (2006), the entire contents of which are incorporated by reference herein. MBPs also may include TALE domains that are engineered to bind methylcytosine in a manner such as described in the following references, the entire contents of each of which are incorporated by reference herein: Tsuji et al., “Modified nucleobase-specific gene regulation using engineered transcription activator-like effectors,” Adv. Drug. Deliv. Rev. 147: 59-65 (2019); Rathi et al., “Engineering DNA backbone interactions results in TALE scaffolds with enhanced 5-methylcytosine selectivity,” Sci. Rep. 7(1): 15067 (2017); and Zhang et al., “Deciphering TAL effectors for 5-methylcytosine and 5-hydroxymethylcytosine recognition,” Nat. Commun. 8(1): 901 (2017).

As used herein, a “fusion protein” is intended to mean an element that includes two or more protein domains with different functional properties (such as enzymatic activity, or that may selectively couple to a target) than one another. The domains may be coupled to one another covalently or non-covalently. A fusion protein may include one or more non-protein elements, such as an epitope or a linker that couples the domains to one another.

As used herein, a “target” is intended to mean an element that selectively, and either covalently or non-covalently, couples to a “target partner.” A target may include an epitope, and a target partner may include a protein or antibody that selectively couples to the epitope. As one example, the SNAP-tag™ protein (commercially available from New England Biolabs, Inc., Ipswitch, MA), may selectively couple to O⁶-benzylguanine and its derivatives. For further details regarding the SNAP-tag™ protein, see Keppler et al., “A general method for the covalent labeling of fusion proteins with small molecules in vivo,” Nature Biotechnology 21(1): 86-89 (2003), the entire contents of which are incorporated by reference herein. As another example, the CLIP-tag™ protein (commercially available from New England Biolabs, Inc., Ipswitch, MA), may selectively couple to O²-benzylcytosine and its derivatives. However, many other pairs of targets and target partners are known in the art and suitably may be used, SpyTag/SpyCatcher, biotin/streptavidin, NTA/His-Tag, and the like.

As used herein, a “linker” is intended to mean an elongated element that couples two other elements to one another. For example, a linker may couple two or more protein domains to one another, or may couple a protein domain to a target. Nonlimiting examples of linkers include polypeptides, or polynucleotides. Nonlimiting examples of polypeptide linkers include GGSGGS (SEQ ID NO: 1), GSSGSS (SEQ ID NO: 2), or the polypeptide linkers listed in the following table:

TABLE
Linker

| Fusion Protein | Type | Sequence# |
|---|---|---|
| scFv | flexible | (GGGGS)₃ (SEQ ID NO: 3) |
| G-CSF-Tf | flexible | (GGGGS)₃ (SEQ ID NO: 3) |
| HBsAg preS1 | flexible | (GGGGS)₃ (SEQ ID NO: 3) |
| Myc-Est2p | flexible | (Gly)₈ (SEQ ID NO: 4) |
| albumin-ANF | flexible | (Gly)₆ (SEQ ID NO: 5) |
| virus coat protein | rigid | (EAAAK)₃ (SEQ ID NO: 6) |
| beta-glucanase- | rigid | (EAAAK)ₙ (n = 1-3) (SEQ ID NO: 6; |
| xylanase | | SEQ ID NO: 7; and SEQ ID NO: 8) |
| hGH-Tf and Tf- | rigid | A(EAAAK)₄ALEA(EAAAK)₄A |
| hGH | | (SEQ ID NO: 9) |
| G-CSF-Tf and | rigid | A(EAAAK)₄ALEA(EAAAK)₄A |
| Tf-G-CSF | | (SEQ ID NO: 9) |
| G-CSF-Tf | flexible | (GGGGS)₃ (SEQ ID NO: 3) |
| G-CSF-Tf | rigid | A(EAAAK)₄ALEA(EAAAK)₄A |
| | | (SEQ ID NO: 9) |
| hGH-Tf | rigid | A(EAAAK)₄ALEA(EAAAK)₄A |
| | | (SEQ ID NO: 9) |
| HSA-IFN-α2b | flexible | GGGGS (SEQ ID NO: 10) |
| HSA-IFN-α2b | rigid | PAPAP (SEQ ID NO: 11) |
| HSA-IFN-α2b | rigid | AEAAAKEAAAKA |
| | | (SEQ ID NO: 12) |
| PGA-tTHS | flexible | (GGGGS)₃ (n = 1, 2, 4) |
| | | (SEQ ID NO: 3; |
| | | SEQ ID NO: 10; SEQ ID NO: 13; |
| | | and SEQ ID NO: 14) |
| interferon- | rigid | (Ala-Pro)ₙ (10-34 aa) (SEQ ID |
| γgpl20 | | NO: 15; SEQ ID NO: 16; SEQ ID |
| | | NO: 17; SEQ ID NO: 18; SEQ ID |
| | | NO: 19; SEQ ID NO: 20; SEQ ID |
| | | NO: 21; SEQ ID NO: 22; SEQ ID |
| | | NO: 23; SEQ ID NO: 24; SEQ ID |
| | | NO: 25; SEQ ID NO: 26; and SEQ |
| | | ID NO: 27) |
| GSF-S-S-Tf | cleavable | disulfide |
| IFN-α2b-HSA | cleavable | disulfide |
| FIX-albumin | cleavable | VSQTSKLTR ↓AETVFPDV^{b} (SEQ |
| | | ID NO: 28) |
| LAP-IFN-1 | cleavable | PLG ↓LWA^{c} (SEQ ID NO: 29) |
| MazE-MazF | cleavable | RVL ↓AEA (SEQ ID NO: 30); |
| | | EDVVCC ↓SMSY (SEQ ID NO: 31); |
| | | GGIEGR ↓GS^{c} |
| | | (SEQ ID NO: 32) |
| Immunotoxins | cleavable | TRHRQPR ↓GWE |
| | | (SEQ ID NO: 33); |
| | | AGNRVRR ↓SVG |
| | | (SEQ ID NO: 34); |
| | | RRRRRRR ↓R ↓R^{d} |
| | | (SEQ ID NO: 35) |
| Immunotoxin | cleavable | GFLG ↓^{e} (SEQ ID NO: 36) |
| G-CSF-Tf and | dipeptide | LE |
| hGH-Tf | rigid | A(EAAAK)₄ALEA(EAAAK)₄A |
| | | (SEQ ID NO: 9) |
| | cleavable | Disulfide |

For further information regarding fusion proteins, and linkers for use in fusion proteins, see Xiaoying Chen, Jennica Zaro, Wei-Chiang Shen, Fusion Protein Linkers: Property, Design and Functionality, Adv. Drug Deliv. Rev. 2013 Oct. 15; 65(10): 1357-1369, the entire contents of which are incorporated by reference herein.

### Compositions and Methods for Detecting Methylcytosine Using a Modified Base Opposite to the Methylcytosine

Provided herein are example assays for cytosine methylation that may be used for targeted, highly multiplexed, quantitative measurement of methylcytosine without the need for upstream enrichment or chemical conversion of cytosine. As described in greater detail below, the present assays may utilize a protein or protein domain that specifically binds methylcytosine in dsDNA, and that may be referred to herein as a methyl-binding protein or MBP. In some examples, the present assays utilize the “base-flipping” property of an MBP to generate a fluorescent signal using the modified base to which the methylcytosine is opposite, e.g., is hybridized; illustratively, an isolated SRA domain may be used without modification to perform such “base-flipping.” In other examples, the present assays may use a fusion protein in which the MBP may be fused to a target partner, the target partner may become coupled to a target that is included in the modified base to which the methylcytosine is opposite, e.g., is hybridized, and a fluorescent signal may be generated based on such coupling; illustratively, an SRA domain fused to a target partner may be used.

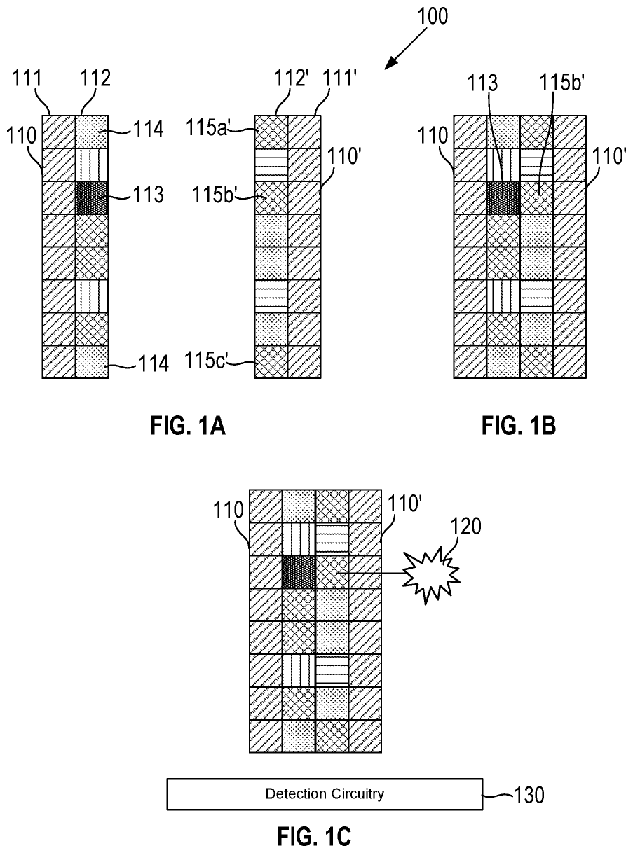

FIGS. 1A-1C schematically illustrate example compositions and operations in a process flow for detecting methylcytosine using a modified base opposite to the methylcytosine. Composition 100 illustrated in FIG. 1A includes first polynucleotide 110 and second polynucleotide 110′, e.g., different fragments of single-stranded DNA or RNA. First polynucleotide 110 may include sugar-phosphate backbone 111 and bases 112, while second polynucleotide 110′ may include sugar-phosphate backbone 111′ and bases 112′. It will be appreciated that first and second polynucleotides 110, 110′ may be significantly longer than is suggested in FIG. 1A, and that the polynucleotides may be in any suitable fluid. It may be desired to assay whether first polynucleotide 110 may include a methylcytosine or a cytosine at a particular location. In the nonlimiting example illustrated in FIG. 1A, first polynucleotide 110 includes methylcytosine 113 and a plurality of cytosines 114, in addition to other bases indicated by shading. In a manner such as provided herein, second polynucleotide 110′ may be used to assay methylcytosine 113 in such a manner that it may be distinguished from cytosines 114, without the need for chemical or other conversion of methylcytosine 113 or of cytosines 114.

For example, second polynucleotide 110′ may include bases 115a′, 115b′ (which may be modified), and 115c′ that are at locations which are complementary to cytosines of first polynucleotide 110. Note that the bases at one or more of these locations of second polynucleotide 110′ may be, but need not necessarily be, complementary to the cytosines of first polynucleotide 110. Illustratively, bases 115a′ and 115c′ may include guanine, while modified base 115b′ may include modified guanine, modified adenine, or any other suitable modified base. Other bases within second polynucleotide 110′ may be complementary to corresponding bases within first polynucleotide 110. In the nonlimiting example illustrated in FIG. 1A, guanine 115a′ may be at a location complementary to cytosine 114, modified base (e.g., modified guanine or other suitable modified base) 115b′ may be at a location complementary to methylcytosine 113, and guanine 115c′ may be at a location complementary to cytosine 114. Modified base 115b′ may be modified for use in obtaining a signal via which it may be determined that the complementary base within first polynucleotide 110 is methylcytosine 113 instead of a cytosine, whereas guanines 115a′ and 115c′ may not be modified for such a purpose. For example, as illustrated in FIG. 1B, first polynucleotide 110 may be hybridized to second polynucleotide 110′ such that modified base 115b′ is opposite to methylcytosine 113. Guanines 115a′, 115b′ may hybridize to respective cytosines 114. In a manner such as illustrated in FIG. 1C, modified base 115b′ may include detectable moiety 120 via which methylcytosine 113 is detectable. That is, detectable moiety 120 may permit the presence of methylcytosine 113 opposite to modified base 115b′ to be determined, as distinguished from the presence of a cytosine opposite to that modified base, and also as distinguished from the presence of cytosines (or methylcytosines) hybridized to guanines 115a′, 115c′.

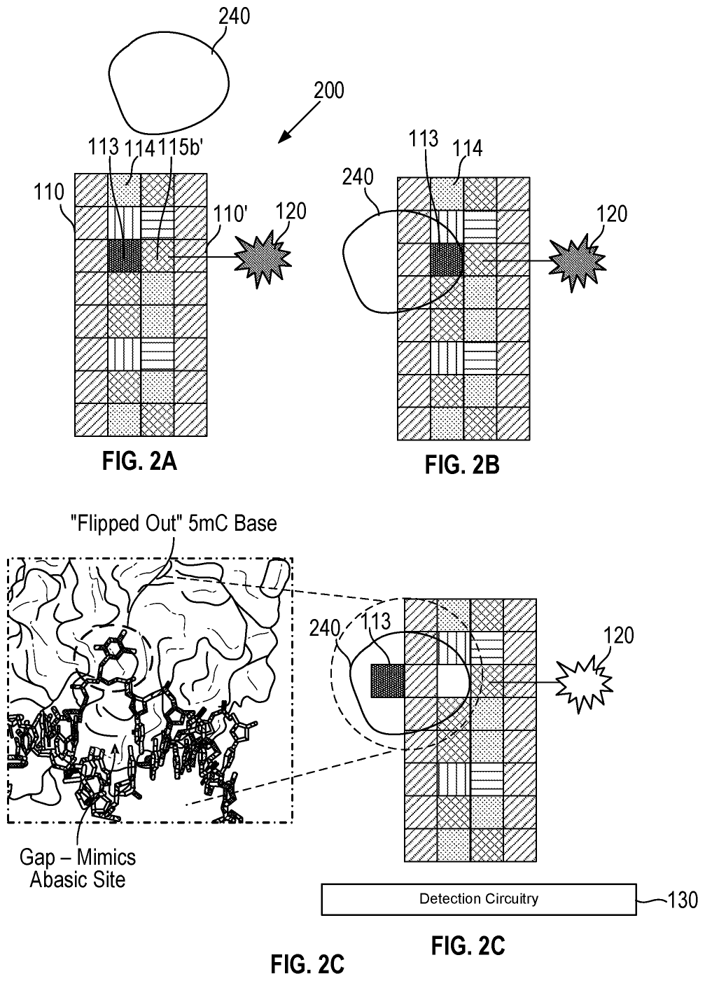

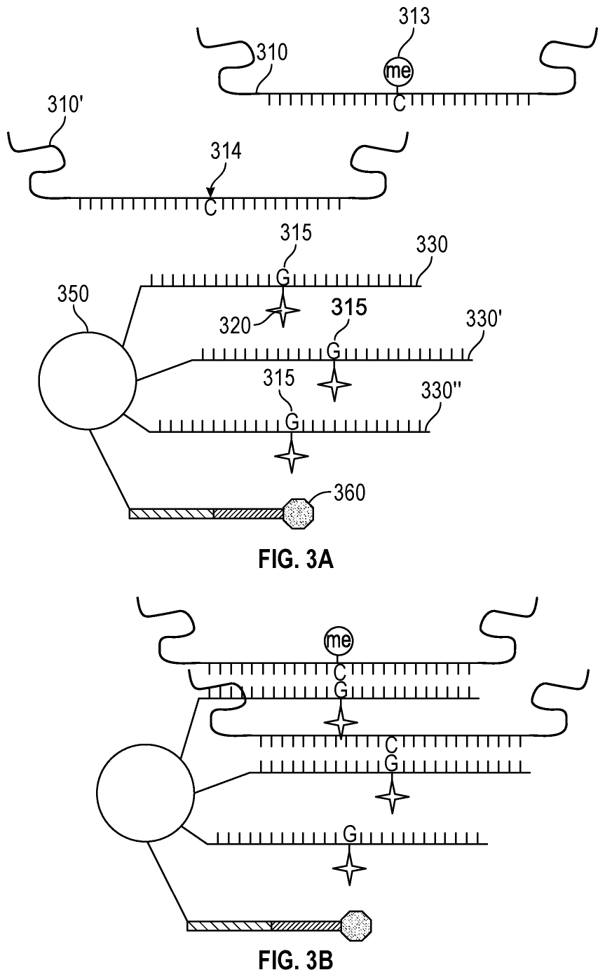

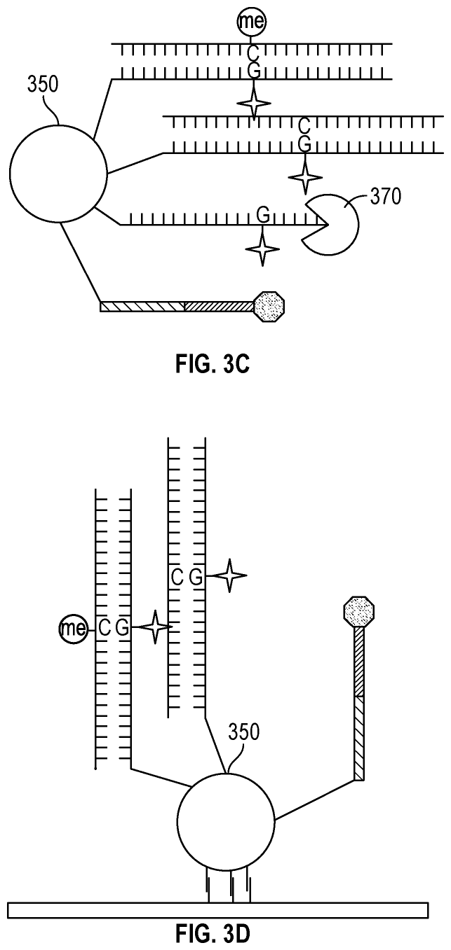

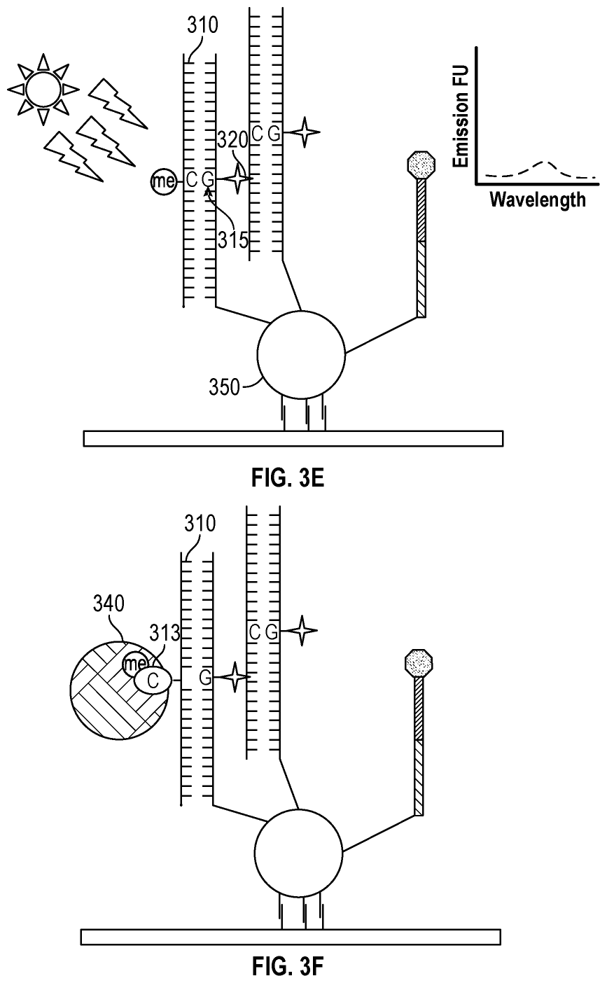

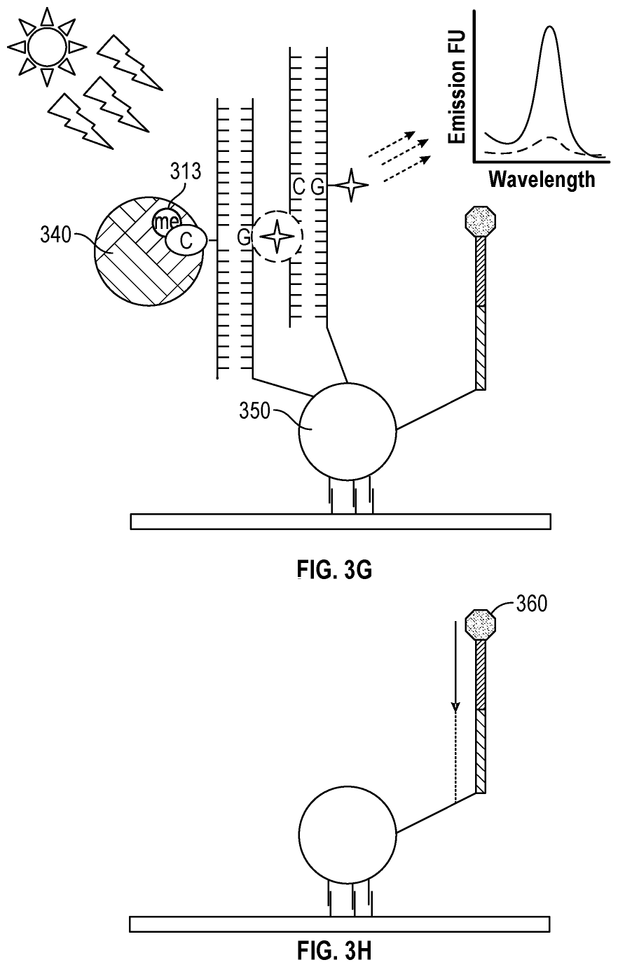

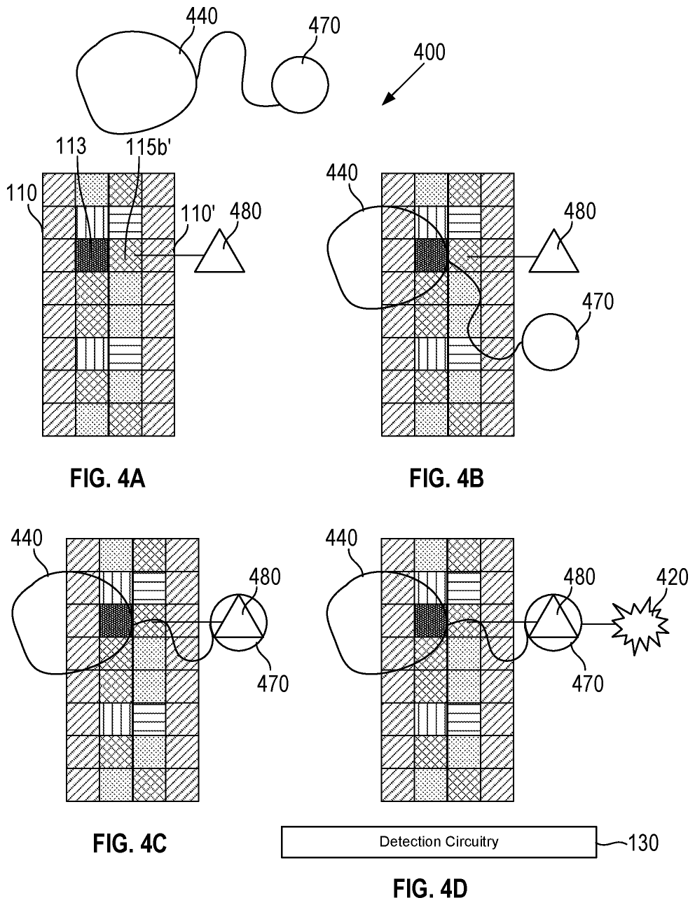

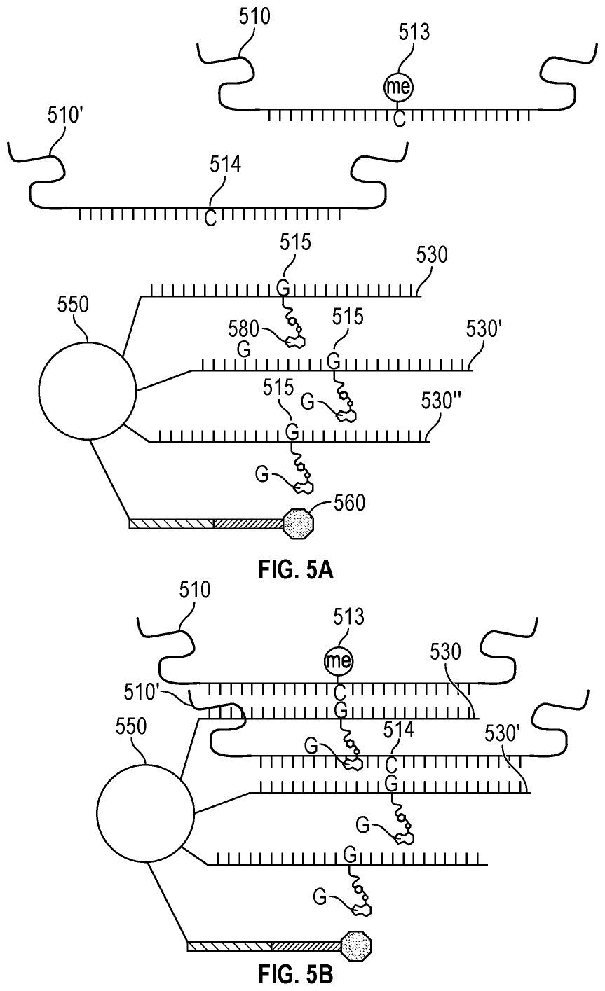

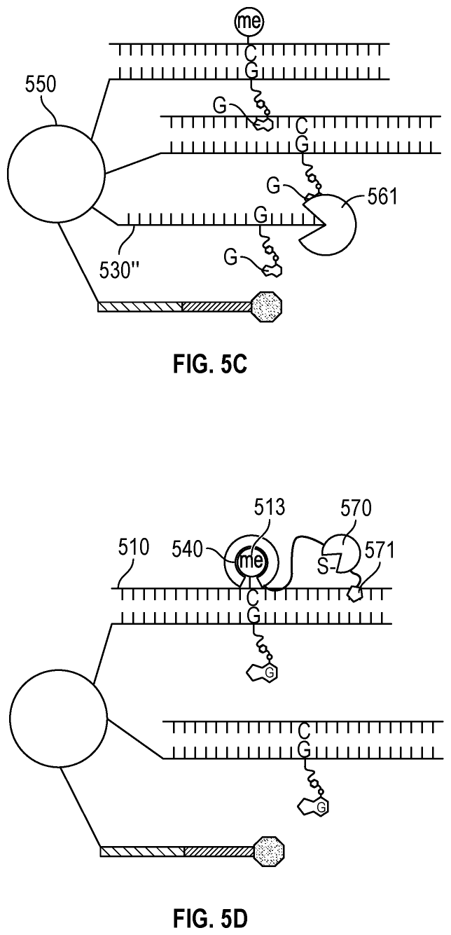

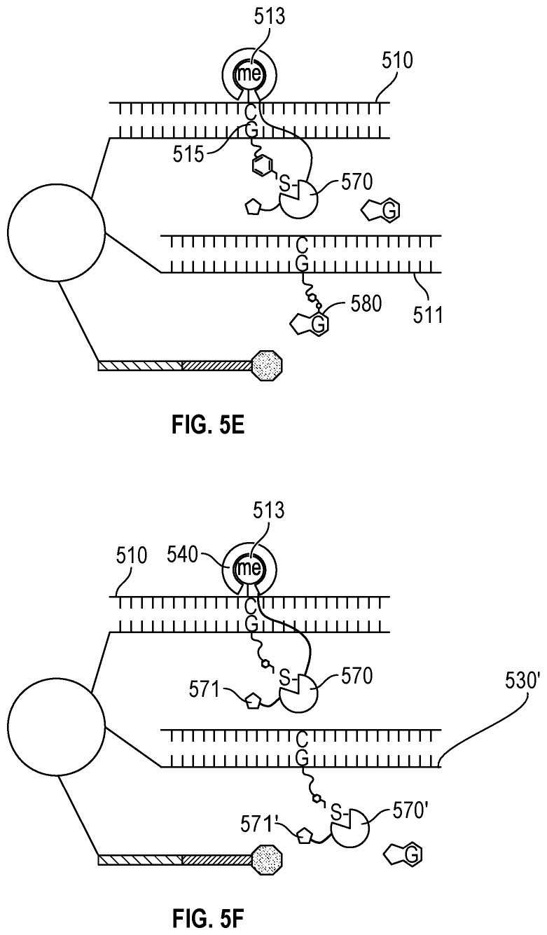

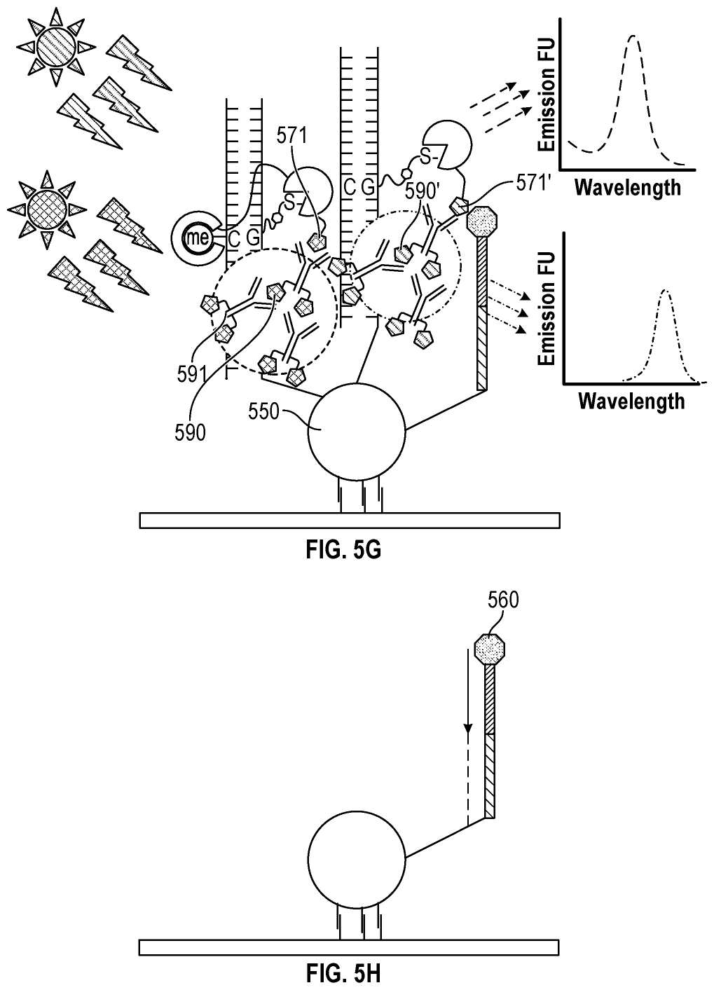

For example, detectable moiety 120 of modified base 115b′ may include a fluorophore, and methylcytosine 113 may be detectable using fluorescence from the fluorophore responsive to excitation light. Illustratively, the fluorescence may be detected using detection circuitry 130. In a manner such as described with reference to FIGS. 2A-2C and 3A-3H, modified base 115b′ may include detectable moiety 120 before second polynucleotide 110′ is hybridized to first polynucleotide 110. Alternatively, in a manner such as described with reference to FIGS. 4A-4D and 5A-5H, modified base 115b′ may be coupled to detectable moiety 120 after second polynucleotide 110′ is hybridized to first polynucleotide 110. It will be appreciated that any suitable method may be used to generate fluorescence in a manner that is correlated to association between modified base 115b′ and methylcytosine 113, as distinguished from hybridization between guanines 115a′, 115c′ and respective cytosines 114.

Optionally, multiple modified bases 115b′ may be used to detect multiple methylcytosines. For example, methylation is often ‘on’ or ‘off’ regionally. For example, CpG islands are regions of genomic DNA where cytosine nucleotides, followed by guanine nucleotides, occur with relatively high frequency. Within such islands, typically all of the CpGs are methylated, or none of them are. Providing a plurality of modified bases 115b′ within second polynucleotide 110′ may facilitate detection of multiple methylcytosines such as may occur in a CpG island. The signals from multiple CpGs in a single strand may be differentiated, for example by using multiple methylcytosine-responsive fluors with non-overlapping excitation or emission wavelengths (e.g., for examples such as described with reference to 2A-2C and FIGS. 3A-3H) or multiple G-linked target moieties with corresponding MBP-fusion proteins (for examples such as described with reference to FIGS. 4A-4D and 5A-5H. For this latter case, it may be useful to do a separate wash and binding step for each target/MBP-fusion pair. In other examples, similar information may be obtained by coupling multiple probe oligonucleotides to a common bead, each with a single modified base at a different position in the respective probe oligonucleotide.

In some examples herein, a protein may be used to induce the fluorescence via which methylcytosine is detected. In some examples, the protein may be coupled to the methylcytosine. For example, FIGS. 2A-2C schematically illustrate example compositions and operations in a process flow for detecting methylcytosine using a modified base including a fluorophore. Composition 200 illustrated in FIG. 2A includes a fluid including protein 240, as well as first and second polynucleotides 110, 110′ hybridized to one another in a manner such as described with reference to FIGS. 1A-1C. Prior to such hybridization, modified base 115b′ (e.g., modified guanine or modified adenine) may include a fluorophore 120, e.g., may itself be or include a solvatochromatic nucleoside, or may include a linker coupling the base to a fluorophore. As suggested in FIG. 2A by the darkened shading, responsive to methylcytosine 113 being located opposite to modified base 115b′, fluorophore 120 fluoresces at a first intensity (which may be zero) and a first wavelength. As illustrated in FIG. 2B, protein 240 may selectively become coupled to methylcytosine 113, e.g., may not become coupled to cytosines 114 or other bases within first polynucleotide 110. Coupling between protein 240 and methylcytosine 113 may dissociate the methylcytosine from modified base 115b′ while first polynucleotide 110 remains hybridized to second polynucleotide 110′. For example, protein 240 may include MBP, illustratively SRA, which causes “base-flipping” of methylcytosine in a manner such as illustrated in FIG. 2C and in greater detail in the inset of FIG. 2C. In nonlimiting examples in which modified base 115b′ includes a modified guanine, such modified guanine may hybridize to methylcytosine 113 when first polynucleotide 110 hybridizes to second polynucleotide 110′, and may become dehybridized from the methylcytosine responsive to base-flipping caused by the MBP.

Responsive to the dissociation of methylcytosine 113 from modified base 115b′ while first polynucleotide 110 remains hybridized to second polynucleotide 110′, fluorophore 120 may fluoresce at a second intensity and a second wavelength, as suggested in FIG. 2C by the lightened shading. The second intensity (with base-flipping) may be different than the first intensity (prior to base-flipping), and accordingly the dissociation—and thus the presence of a methylcytosine—may be detected via such change in intensity as detected by detection circuitry 130. Additionally, or alternatively, the second wavelength (with base-flipping) may be different than the first wavelength (prior to base-flipping), accordingly the dissociation—and thus the presence of a methylcytosine—may be detected via such change in wavelength as detected by detection circuitry 130.

Modified base 115b′ (e.g., modified guanine or other modified base) may include any suitable fluorophore 120 such that the wavelength and/or intensity changes of that fluorophore responsive to dissociation of methylcytosine 113 from that modified base. In some examples, modified base 115b′ (e.g., modified guanine or modified adenine) may include a solvatochromatic nucleoside. Accordingly, although FIGS. 1A-1C, 2A-2C, and other figures herein may appear to suggest that the fluorophore 120 is separate from and coupled to the modified base, it should be appreciated that the modified base may itself be the fluorophore. Nonlimiting examples of solvatochromatic nucleosides, and other suitable fluorophores that may be included within the present modified bases, are provided elsewhere herein.

FIGS. 3A-3H schematically illustrate additional example compositions and operations in a process flow for detecting methylcytosine using a modified base that includes a fluorophore. This process flow similarly uses a fluorophore to generate a fluorescence signal responsive to selective action of a protein upon a methylcytosine. As illustrated in FIG. 3A, a plurality of single-stranded polynucleotide (e.g., genomic DNA) fragments 310, 310′ are brought into contact with a substrate 350, such as a bead, to which a plurality of identical probe oligonucleotides 330, 330′, 330″ are coupled. Such contact may be within a suitable fluid (not specifically illustrated). Fragments 310, 310′ may have any suitable length, and in some examples may have approximately the same length as that of probe oligonucleotides 330, 330′, 330″. Each of probe oligonucleotides 330, 330′, 330″ may include a modified base 315 (e.g., modified guanine) including fluorophore 320, at a location within its sequence at which it is desired to detect whether a cytosine within the polynucleotide fragments is methylated or not (one of such fluorophores being expressly labeled for simplicity). Generating the single-stranded genomic polynucleotide fragments 310, 310′ may include fragmenting double-stranded polynucleotides, and then heating the resulting double stranded fragments in the presence of the bead-linked oligonucleotides 330, 330′, 330″ so as to render them single-stranded.

As illustrated in FIG. 3B, fragment 310 (including methylcytosine 313 at the location being assayed) may become hybridized to a first one of the probe oligonucleotides 330, while fragment 310′ (including unmethylated cytosine 314 at the location being assayed) may become hybridized to a second one of the probe oligonucleotides 330′. Following such capture, beads 350 may be washed or otherwise processed to remove unbound fragments. Optionally, in a manner such as illustrated in FIG. 3C, a DNAse digest may be performed with a ssDNA specific exonuclease 370 (e.g., E. coli exonuclease I) to reduce background signal from unbound probe oligonucleotides (e.g., 330″ as illustrated in FIG. 3A) on bead 350. In a manner such as illustrated in FIG. 3D, the beads then may be loaded onto a flowcell, e.g., using standard surface chemistry or a bead-capture surface to specifically trap the beads.

In a manner such as illustrated in FIG. 3E, a background fluorescence scan with excitation and emission wavelengths appropriate for fluorophore 320 may be performed. This may establish a background of fluorescence for bead 350 in the absence of a protein for dissociating the methylcytosine from modified base 315. For example, as illustrated in FIG. 3F, protein 340 selectively may be coupled to methylcytosine 313 of fragment 310, e.g., following application to the flowcell of a binding solution including a plurality of proteins 340. Following such coupling, mild wash steps may be used to reduce any nonspecific coupling of proteins 340. Note that other proteins 340 in the solution may become bound to other methylcytosines that may be present in other locations of fragment 310, and/or that may be present in fragment 311. However, because the particular protein 340 illustrated in FIG. 3F is coupled to the methylcytosine 313 which is opposite to modified base 315, which in turn includes fluorophore 320, the binding of that protein may cause fluorophore 320 to generate a detectable signal from which the presence of methylcytosine 313 may be determined (e.g., because protein 340 dissociates the methylcytosine from the modified base, causing a change in intensity and/or wavelength of the fluorophore). For example, as illustrated in FIG. 3G, bead 350 may be scanned again while protein 340 is coupled to methylcytosine 313, which may generate a fluorescent signal. The amplitude of such signal (solid line of plot) may be compared to the background (dotted line of plot), and their difference in intensities may be proportional to the amount of methylcytosine 313 that is present at the location opposite to that of modified base 315 in the fragments being assayed.

Although FIGS. 3A-3G may focus on interactions between particular polynucleotide fragments and a particular bead, it will be appreciated that bead 350 having probe oligonucleotides 330, 330′, 330″ may be one of a plurality of beads respectively having other probe oligonucleotides coupled thereto that similarly have sequences with modified bases at locations at which it is desired to detect whether cytosine in polynucleotide fragments is methylated or not. As illustrated in FIGS. 3A-3H, a decode oligonucleotide 360 also may be coupled to substrate 350, and which may be read (e.g., using sequencing by synthesis or hybridization of a fluorescently labeled oligonucleotide) to identify the particular probe oligonucleotides 330, 330′, 330″ coupled to bead 350 in a manner such as illustrated in FIG. 3H. Note that decode oligonucleotide 360 may be protected on the 3′ end so as to inhibit its degradation by the exonuclease, and that decode oligonucleotides 360 coupled to different beads may include common sequences at which a primer may land and extend from for use in determining the sequence of the decode oligonucleotide. The sequence of the decode oligonucleotide can be used to determine the locus being assayed using the probe oligonucleotides coupled to the respective bead, and/or may be used for sample indexing. For further details regarding use of a decode oligonucleotide, see Gunderson et al., “Decoding Randomly Ordered DNA Arrays,” Genome Research 14: 870-877 (2004), the entire contents of which are incorporated by reference herein.

Referring again to FIGS. 1A-1C, it will be appreciated that other methods may be used to generate fluorescence in a manner that is correlated to methylcytosine 113 being opposite to a modified base 115b′, as distinguished from respective cytosines 114 hybridizing to guanines 115a′ and 115c′. In examples such as described with reference to FIGS. 2A-2C and 3A-3H, a protein may induce the fluorescence, e.g., by dissociating methylcytosine from the modified base in such a manner as to induce or alter fluorescence from a fluorophore included in the modified base. Alternatively, in examples such as now will be described with reference to FIGS. 4A-4D and 5A-5H, the protein instead may be coupled to another protein.

For example, FIGS. 4A-4D schematically illustrate example compositions and operations in a process flow for detecting methylcytosine using a modified base including a target to which a fluorophore may be coupled. Composition 400 illustrated in FIG. 4A includes a fluid including first protein 440 coupled to second protein 470 (e.g., via a linker). The first and second proteins 440, 470 may, in some examples, include different parts of a fusion protein. The fluid also may include as first and second polynucleotides 110, 110′ hybridized to one another in a manner such as described with reference to FIGS. 1A-1C. Prior to such hybridization, modified base 115b′ (e.g., modified guanine or modified adenine) may include a first target 480 (e.g., via a linker) to which second protein 470 selectively binds when first protein 440 couples to the methylcytosine. As illustrated in FIG. 4B, and in a manner similar to that described elsewhere herein, protein 440 may selectively become coupled to methylcytosine 113. Such coupling may place second protein 470 sufficiently close to first target 480 as to promote coupling between the two in a manner such as illustrated in FIG. 4C. Fluorophore 420, illustrated in FIG. 4D, may be coupled to second protein 470, or to target 480, or to both second protein 470 and target 480. In nonlimiting examples such as described with reference to FIGS. 4C-4D, fluorophore 420 may be formed by coupling a first fluorophore component, which is coupled to second protein 470, to a second fluorophore component, which is coupled to target 480. Fluorescence from fluorophore 420 may be detected by detection circuitry 130, and thus the presence of methylcytosine 113′ opposite to modified base 115b′ (as illustrated in FIG. 2B) may be detected.

In examples in which fluorophore 420 (or component thereof) is coupled to the second protein 470, such coupling may be performed or after first protein 440 becomes coupled to methylcytosine 113. Similarly, in examples in which fluorophore 420 (or component thereof) is coupled to target 480, such coupling may be performed before or after target 480 is included in modified base 115b′. For example, second protein 470 or target 480 may include a second target (not specifically illustrated), and fluorophore 420 may be coupled to a third protein (not specifically illustrated) that selectively binds to the second target. Illustratively, the second target may include an epitope, and the third protein may include an antibody. The antibody may include fluorophore 420.

It will be appreciated that any suitable second protein 470 (target partner) and any suitable target 480 may be used that selectively may couple to one another. For example, second protein 470 may include a SNAP protein and target 480 may include an O-benzylguanine. Or, for example, second protein 470 may include a CLIP protein and target 480 may include an O-benzylcytosine. Or, for example, second protein 470 may include SpyTag and target 480 may include SpyCatcher. Or, for example, second protein 470 may include SpyCatcher and target 480 may include SpyTag. Or, for example, second protein 470 may include biotin and target 480 may include streptavidin. Or, for example, second protein 470 may include streptavidin and target 480 may include biotin. Or, for example, second protein 470 may include NTA and target 480 may include His-Tag. Or, for example, second protein 470 may include His-Tag and target 480 may include NTA. Other suitable combinations of proteins and targets readily may be envisioned.

FIGS. 5A-5H schematically illustrate additional example compositions and operations in a process flow for detecting methylcytosine using a modified base that includes a target to which a fluorophore may be coupled. In this process flow, a first protein-second protein-epitope fusion protein (e.g., an MBP-SNAP tag-epitope fusion protein) may be used to covalently link the first protein, and the fluorophore, to the modified base based on a methylcytosine being opposite to that modified base. Such a process flow may readily be adapted to promote amplification of the fluorescent signal and/or may be adapted to allow for quantitation of methylation site stoichiometry. Additionally, such a process flow may allow for protein binding steps to be performed prior to loading beads onto a flowcell.

As illustrated in FIG. 5A, a plurality of single-stranded polynucleotide (e.g., genomic DNA) fragments 510, 510′ are brought into contact with a substrate 550, such as a bead, to which a plurality of identical probe oligonucleotides 530, 530′, 530″ are coupled. Such contact may be within a suitable fluid (not specifically illustrated). Fragments 510, 510′ may have any suitable length, and in some examples may have approximately the same length as that of probe oligonucleotides 530, 530′, 530″. Each of probe oligonucleotides 530, 530′, 530″ may include modified base 515 (such as a modified guanine or adenine), including target 580, at a location within its sequence at which it is desired to detect whether a cytosine within the polynucleotide fragments is methylated or not. Generating the single-stranded polynucleotide fragments 510, 510′ may include fragmenting double-stranded polynucleotides, and then heating the resulting double stranded fragments in the presence of the bead-linked oligonucleotides 530, 530′, 530″ so as to render them single-stranded.

As illustrated in FIG. 5B, fragment 510 (including methylcytosine 513 at the location being assayed) may become hybridized to a first one of the probe oligonucleotides 530, while fragment 510′ (including unmethylated cytosine 514 at the location being assayed) may become hybridized to a second one of the probe oligonucleotides 530′. Following such capture, beads 550 may be washed or otherwise processed to remove unbound fragments. Optionally, in a manner such as illustrated in FIG. 5C, a DNAse digest may be performed with a ssDNA specific exonuclease 561 (e.g., E. coli exonuclease I) to reduce background signal from unbound probe oligonucleotides (e.g., 530″) on bead 550. Such digestion may be particularly useful in examples in which stoichiometry is desired to be assayed.

As illustrated in FIG. 5D, the first protein-second protein-epitope fusion protein (e.g., MBP-SNAP tag-epitope fusion protein) 540, 570, 571 selectively may be coupled to methylcytosine 513 of fragment 510, e.g., within a solution including a plurality of the fusion proteins. As illustrated in FIG. 5E, second protein 570 may become coupled to target 580 (e.g., O-benzylguanine). Note that other fusion proteins in the solution may become bound to other methylcytosines that may be present in other locations of fragment 510, and/or that may be present in fragment 511. However, because the particular fusion protein illustrated in FIG. 5E is coupled to the methylcytosine 513 which is opposite to modified base 515, which in turn includes target 580, the binding of the second protein 570 of the fusion protein to that target may provide a handle (epitope 571 as illustrated in FIG. 5F) to which a fluorophore selectively may be coupled for use in generating a detectable signal from which the presence of methylcytosine 513 may be determined. For example, the target 580 included in the modified base of probe oligonucleotide 530′ may not become coupled to the second protein 570 of any of the fusion proteins, because such second proteins are not held in proximity to such target 580 via coupling of the first protein 540 (illustrated in FIG. 5D) to a methylcytosine. Optionally, in a manner such as illustrated in FIG. 5F, the target 580 included in the modified base of probe oligonucleotide 530′ (and any other targets that are not coupled to a respective second protein 570) may be coupled to an alternative second protein 570′ to which an alternative epitope 571′ is coupled. The alternative second protein 570′ may be the same type of protein as second protein 570, but epitope 571′ may be different than epitope 571 so as to facilitate distinguishing methylcytosine (to which epitope 571 is indirectly coupled) from cytosine (to which epitope 571′ is indirectly coupled.

In a manner such as illustrated in FIG. 5G, the beads then may be loaded onto a flowcell, e.g., using standard surface chemistry or a bead-capture surface to specifically trap the beads. Fluorophore-labeled antibodies 590 that recognize epitope 571 are added and allowed to bind the epitopes that are covalently linked to the methylcytosine via the first and second proteins 540, 570 (as illustrated in FIG. 5C). Signal amplification may be performed, e.g., using secondary antibodies 591 that are raised against the IgG matching the primary antibody 590 or that recognizes a hapten conjugated to the primary antibody and itself. Optionally, fluorophore-labeled antibodies 590′ that recognize epitope 571′ are added and allowed to bind the epitopes that are linked to unmethylated cytosines via alternative second proteins 570′. Signal amplification may be performed, e.g., using secondary antibodies 591′ that are raised against the IgG matching the primary antibody 590′ or that recognizes a hapten conjugated to the primary antibody and itself. The fluorophores of antibodies 590, 591 may emit light at a different wavelength than the fluorophores of antibodies 590′, 591′. As illustrated in FIG. 5G, bead 550 may be scanned while antibodies 590, 591 are coupled to the target 580 coupled to methylcytosine, and antibodies 590′, 591′ optionally are coupled to the target 580 coupled to unmethylated cytosine. As such, fluorescent signals of two different wavelengths may be generated (or one wavelength if unmethylated cytosines are not fluorescently labeled). The intensity of the fluorescence from antibodies 590, 591 is proportional to the number of first protein-second protein-epitope fusion proteins that are coupled to methylcytosines that are opposite modified bases including targets 580 (as illustrated in FIGS. 5E and 5F. The intensity of the fluorescence from optional antibodies 590′, 591′ is proportional to the number of second protein-epitope fusion proteins that are coupled to unmethylated cytosines that are opposite modified bases including targets 580. For quantitation of stoichiometry (the proportion of methylcytosine to unmethylated cytosine at the assayed location), the intensity of the fluorescence from antibodies 590, 591 may be divided by (or otherwise compared to) the intensity of fluorescence from optional antibodies 590′, 591′. For example, the stoichiometry of meC to C may be determined by the ratio of fluorescence from 590, 591 to that of 590′, 591.

Although FIGS. 5A-5G may focus on interactions between particular polynucleotide fragments and a particular bead, it will be appreciated that bead 550 having probe oligonucleotides 530, 530′, 530″ may be one of a plurality of beads respectively having other probe oligonucleotides coupled thereto that similarly have sequences with modified bases at locations at which it is desired to detect whether cytosine in polynucleotide fragments is methylated or not. As illustrated in FIGS. 5A-5G, a decode oligonucleotide 560 also may be coupled to substrate 550, and which may be read (e.g., using sequencing by synthesis or hybridization of a fluorescently labeled oligonucleotide) to identify the particular probe oligonucleotides 530, 530′, 530″ coupled to bead 550 in a manner such as illustrated in FIG. 5H.

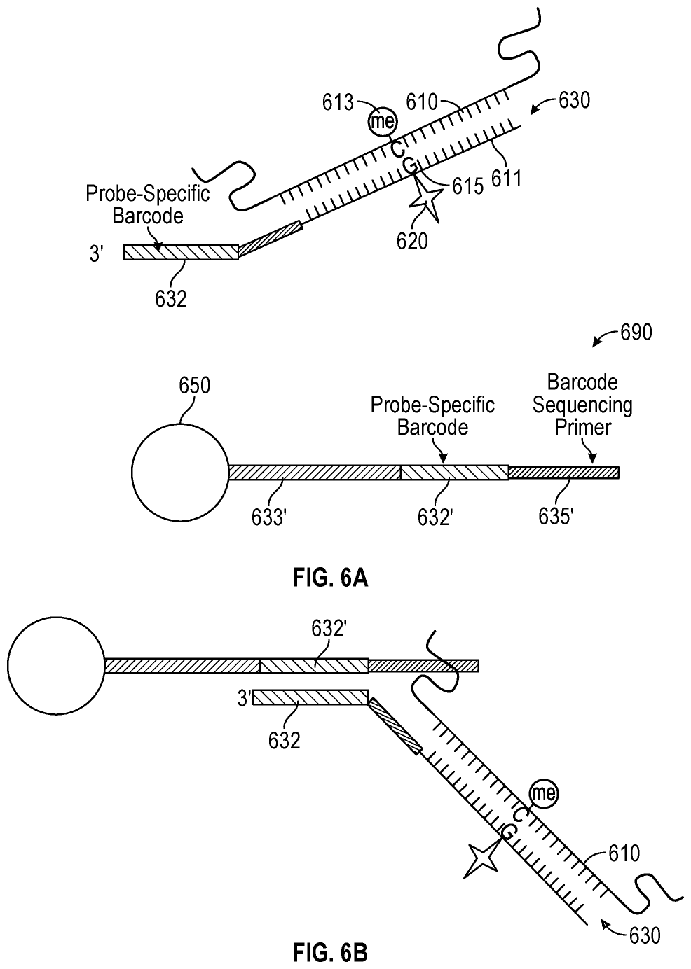

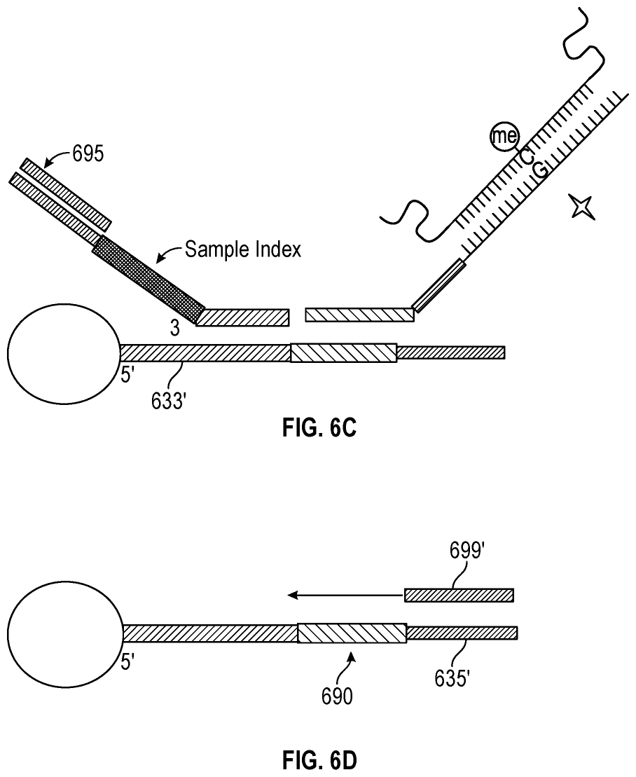

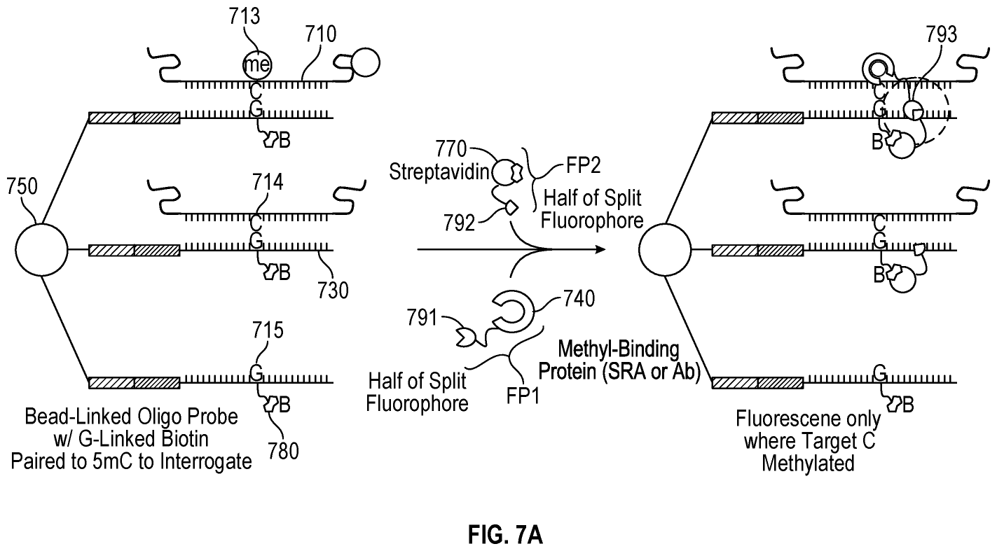

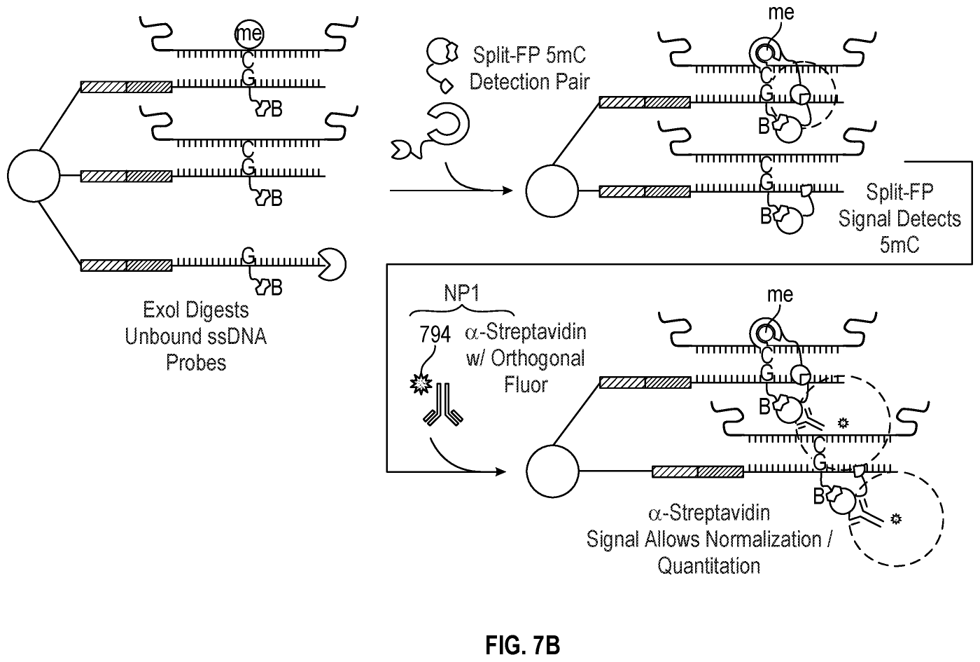

It will be appreciated that any suitable fluorophore may be used to detect methylcytosine in any suitable manner. For example, FIGS. 7A-7B schematically illustrate example compositions and operations in a process flow for detecting methylcytosine using a modified base opposite to the methylcytosine. Referring first to FIG. 7A, first fusion protein FP1 may include a MBP 740 (e.g., the UHRF1 SRA domain), which is coupled to one half 791 of a split fluorescent protein, and a second fusion protein FP2 may include the complementary half 792 of the split fluorescent protein included in FP1, which is coupled to a target partner 770 that selectively may be coupled to target 780 that is attached to a modified base opposite to a methylcytosine in a manner similar to that described with reference to FIGS. 4A-4D and 5A-5H. The modified base may be provided within a probe oligonucleotide 730 that may be at least partially complementary to a target polynucleotide 710 of interest, where the modified base (e.g., modified guanine or adenine) is opposite to the cytosine to be assayed for methylation, e.g., in a manner similar to that described with reference to FIGS. 4A-4D and 5A-5H. The probe oligonucleotide 730 may be directly anchored to a solid support such as a paramagnetic bead 750 (where all oligos on a single bead target the same DNA sequence in a manner such as described with reference to FIG. 2A-2C, 3A-3H, 4A-4D, or 5A-5H), or could contain a secondary capture sequence for in-solution binding followed by bead capture (e.g., in a manner such as described elsewhere herein, e.g., with reference to FIGS. 6A-6D or as described in International Patent Application No. PCT/EP2020/078653). Target multiplexing may be achieved by including multiple bead types in the binding reaction, each with a unique decode oligo that can be identified after imaging of methylation fluorescence by on-bead SBS in a manner such as described elsewhere herein.

An example workflow using this system may include target DNA binding. For example, following an optional fragmentation step, a first oligonucleotide (e.g., target DNA) 710 may be denatured in the presence of probe oligo(s) 730, then re-annealed to allow binding of the first oligonucleotide the probe oligo as illustrated in FIG. 7A. This will allow the cytosines 713, 714 at selected locations within the first oligonucleotide 710 to be assayed for methylation, by placing those cytosines opposite to modified bases 715 which include the target 780 for which FP2's target partner is selective. As illustrated in FIG. 7A, the example workflow may include adding fusion proteins FP1 and FP2 to the solution containing the target-capture oligo duplex. At positions where the target contains a methylated cytosine 713 opposite the modified base 715, the MBP 740 of FP1 binds to the methylated cytosine, and the target partner 770 of FP2 binds to the target 780 attached to the modified base. This brings the two halves 791, 792 of the split fluorescent protein into proximity with one another, allowing formation of an active fluorophore 793 as illustrated in FIG. 7A. On target fragments where the cytosine 714 in the target position is not methylated, the target partner 770 of FP2 still binds to the modified base 715 in the probe oligo 730. However, the half 792 of the split fluorophore which is included in FP2 will not be held in proximity to the half 791 of the split fluorophore (because there is no methylcytosine to be bound by MBP 740 of FP1), thus inhibiting formation of an active fluorophore.

As illustrated in FIG. 7A, the example workflow may include imaging. For example, beads 750 (or other solid support) with bound target-capture oligos, FP1 and FP2 may be imaged in such a way that the fluorescent signal from each bead, containing multiple oligos targeting a single methylated DNA fragment, can be distinguished from other beads in the field of view. An example of this would be a flowcell treated to allow capture and anchoring of beads on the flowcell surface. The example workflow further may include decoding. For example, in a manner such as described elsewhere herein, decode oligonucleotides unique to each bead type (and therefore target DNA sequence) are read out using on-bead SBS (or other decoding methods). This allows the fluorescent signal collected the imaging operation to be linked to a specific DNA target.

Although the workflow as outlined with reference to FIG. 7A may not necessarily include an explicit normalization step (e.g., generation of a signal proportional to the number of methylated or non-methylated cytosines), a number of normalization methods may be used. For example, as illustrated in FIG. 7B, one option would be to include a second fluorophore 794, with orthogonal spectral characteristics to the split fluorescent protein in FP1 and FP2, attached to a protein NP1 that binds FP2 either through native epitopes (e.g., an anti-FP2 antibody) or by a small-molecule or peptide target covalently attached to FP2 in a manner similar to that described with reference to FIGS. 5A-5H. Following target DNA binding, the sample may be treated with a ssDNA-specific exonuclease to remove any unbound capture oligos, treated with FP1, FP2, and the NP1 during step 2. Imaging of the NP1 fluorophore may provide a measure of the ‘total’ target oligonucleotides 730 (with or without methylcytosine) bound to capture probes that may be used to normalize the fluorescent signal from FP1-FP2.

Nonlimiting examples of split fluorescent proteins may be found in the following references, the entire contents of each of which are incorporated by reference herein: Tamura et al., “Multiplexed Labeling of Cellular Proteins with Split Fluorescent Protein Tags.” Communications Biology 4(1): 257 (2021); Cabantous et al., “Protein Tagging and Detection with Engineered Self-Assembling Fragments of Green Fluorescent Protein.” Nature Biotechnology 23(1): 102-7 (2005); Feng et al., “Improved Split Fluorescent Proteins for Endogenous Protein Labeling.” Nature Communications 8(1): 370 (2017); Kamiyama et al., “Versatile Protein Tagging in Cells with Split Fluorescent Protein.” Nature Communications 7 (March): 11046 (2016); Pedelacq et al., “Development and Applications of Superfolder and Split Fluorescent Protein Detection Systems in Biology.” International Journal of Molecular Sciences 20 (14), https://doi.org/10.3390/ijms20143479 (2019); and Romei et al., “Split Green Fluorescent Proteins: Scope, Limitations, and Outlook,” Annual Review of Biophysics 48 (May): 19-44 (2019).

It will be appreciated that the split fluorescent protein may take any suitable form, and that halve 791 may have any suitable structure for inducing a detectable signal (e.g., fluorescence) in halve 792 responsive to proximity between halves 791 and 792, or that halve 792 may have any suitable structure for inducing a detectable signal (e.g., fluorescence) in halve 791 responsive to proximity between halves 791 and 792. In one nonlimiting example, one of half 791 or half 792 may include a portion of a split horseradish peroxidase (HRP) protein, and the other of half 791 or half 792 may include the other portion of the split HRP protein. The supernatant may include a reagent (such as a tyramide reagent) that provides for colorimetric detection or fluorescent signal amplification of the split HRP protein, when the two portions of the split HRP protein join with each other. For example, the enzymatic activity of the joined HRP protein may activate the tyramide fluor in solution, which then covalently attaches to nearby proteins. For further details regarding a split HRP protein, see Martell et al., “A Split Horseradish Peroxidase for the Detection of Intercellular Protein-Protein Interactions and Sensitive Visualization of Synapses.” Nature Biotechnology 34 (7): 774-80 (2016), the entire contents of which are incorporated by reference herein. For further details regarding reagents for colorimetric detection or fluorescent signal amplification of a split HRP protein, see the following references, the entire contents of each of which are incorporated by reference herein: Bobrow et al., “Catalyzed Reporter Deposition, a Novel Method of Signal Amplification. Application to Immunoassays.” Journal of Immunological Methods 125(1-2): 279-85 (1989); and Earnshaw et al., “Signal Amplification in Flow Cytometry Using Biotin Tyramine.” Cytometry 35(2): 176-79 (1999).

It will be appreciated that any suitable protein(s) may be used to detect methylcytosine opposite to a modified base. For example, proteins 240, 340, 440, 540, or 740 may include a methyl binding protein (MBP). A nonlimiting example of MBP is a SET and Ring finger Associated (SRA) domain, although other MBPs suitably may be used.

In certain examples such as described with reference to FIGS. 1A-1C, 2A-2C, 3A-3H, 4A-4D, 5A-5H, and 7A-7B, the second polynucleotide (including the modified base opposite to the methylcytosine being detected) may be directly coupled to a substrate, such as a bead. However, it will be appreciated that the present compositions and methods readily may be adapted for use with second polynucleotides that are not directly coupled to a substrate. Illustratively, the present compositions and methods may be adapted for use in a manner similar to that described in International Patent Application No. PCT/EP2020/078653, filed on Oct. 12, 2020 and entitled “Systems and Methods for Detecting Multiple Analytes,” the entire contents of which are incorporated by reference herein.

For example, FIGS. 6A-6D schematically illustrate additional example compositions and operations in a process for detecting methylcytosine using a modified base opposite to which the methylcytosine. As illustrated in FIG. 6A, first polynucleotide 610 may hybridize in solution to probe oligonucleotide 630 including second polynucleotide 611. Such hybridization may enrich for first polynucleotide 610 by pulling it out of solution using probe oligonucleotide 630 during an enrichment step, for example using hybridization between oligonucleotide 632′ coupled to oligonucleotide 690 and oligonucleotide 632 coupled to probe oligonucleotide 630. Both probe oligonucleotide 630 and first 610 may be coupled to the bead 650 as a result of the pulldown. Following pulldown, any polynucleotide fragments that do not include oligonucleotide 610, and thus do not hybridize to second polynucleotide 611 of probe oligonucleotide, may be washed away. First polynucleotide 610 may include methylcytosine 613 at a location being assayed. Second polynucleotide 611 may include modified base 615 (e.g., modified guanine or modified adenine) including a fluorophore or other detectable moiety 620 or to which such fluorophore or other detectable moiety 620 otherwise may be directly coupled or may be indirectly coupled in a manner similar to that described with reference to FIGS. 1A-1C, 2A-2C, 3A-3H, 4A-4D, 5A-5H, and 7A-7B, as well as a probe-specific barcode 632 in a manner similar to that described in International Patent Application No. PCT/EP2020/078653. Probe oligonucleotide 630 may not be coupled to a substrate (e.g., a bead), but instead may be provided in solution and incubated with first polynucleotide 610 before probe oligonucleotide 630 is coupled to bead 650. Bead 650 may be separately provided (e.g., not initially coupled to probe oligonucleotide 630), and may be directly coupled to third oligonucleotide 690. Third oligonucleotide 690 may include probe-specific barcode 632′ (a code identifying first polynucleotide 610), as well as and barcode sequencing primer 635.

In a manner such as illustrated in FIG. 6B, the probe specific barcode 632 of probe oligonucleotide 630, to which first polynucleotide 610 may be hybridized in an earlier operation, may hybridize to probe specific barcode 632′ in a manner so as to indirectly couple first polynucleotide 610 to bead 650. As such, third oligonucleotide 690 may be coupled to substrate 650 separately from second polynucleotide 611, and may couple the second polynucleotide to the substrate. Before or after hybridization of probe specific barcodes 632, 632′ to one another, substrate 650 may be coupled to a flowcell in a manner such as described with reference to FIG. 3D or 5H. Methylcytosine 613 then may be assayed using modified base 615 in a manner such as provided herein, e.g., by detecting a fluorescent signal in a manner such as described with reference to FIG. 2A-2C, 3A-3H, 4A-4D, 5A-5H, or 7A-7B. Detecting methylcytosine 613 using modified base 615 may include identifying the first polynucleotide 610 using probe-specific barcode 632 (the code). For example, as illustrated in FIG. 6C, a fourth oligonucleotide 695 may be hybridized to region 633′ of third oligonucleotide 690 and sequenced. In one nonlimiting example, region 633′ may be used as a sample index for use in multiplexing different samples with one another. For example, barcode 632 (illustrated in FIG. 6A) may be probe specific, enabling each assayed region to be identified. Oligo 695 may correspond to the particular sample, and may be annealed to every bead and every probe barcode from a particular sample, and thus may be used to identify that each of those beads correspond to a particular sample. Other oligos 695 may be annealed to other beads from other samples. The various samples may be combined together on the sequencer, and the oligos 695 may be used to deconvolve which bead came from each sample.

In one example workflow, a probe specific barcode 632 may be added to the sample and may bind regions of interest. The bead may be pre-hybridized with probe 650 with the sample index, or such operation may be performed concurrently with binding of probe specific barcode 632, or such operation may be performed after obtaining the complex illustrated in FIG. 6B. The resulting complex may be loaded onto a sequencer, and fluorescence measured. The sample index primer and sequence sample index may be annealed. The complex may be denatured and then decoded in a manner such as described with reference to FIG. 6D.

As illustrated in FIG. 6D, probe oligonucleotide 630 may be dehybridized from oligonucleotide 690 (see FIG. 6B), primer 699′ may be hybridized to barcode sequencing primer region 635′ of oligonucleotide 690, and probe-specific barcode 632′ may be sequenced using sequencing-by-synthesis (SBS). From the sequence of probe-specific barcode 632′, it may be determined which particular probe oligonucleotide 630 hybridized to substrate 650. From this information it is known at which location methylation was being assayed via modified base 615, and from the fluorescent signal it may be determined whether the cytosine at that location was methylated. Note that probe-specific barcode 632′ may be used to assay multiple targets simultaneously, in a multiplexed workflow. For example, for any given sample, many different regions may be assayed for methylcytosine, and the corresponding probe-specific barcodes sequenced to determine the results of assaying which respective regions.

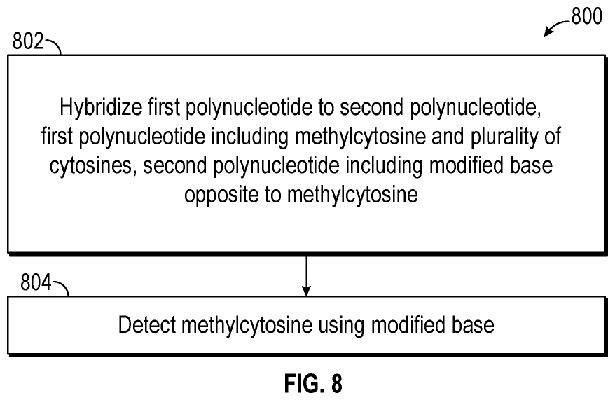

It will be appreciated that compositions such as described with reference to FIGS. 1A-1C, 2A-2C, 3A-3H, 4A-4D, 5A-5H, 6A-6D, and 7A-7B are purely illustrative, and that any other suitable compositions may be used in a method for detecting methylcytosine. FIG. 8 illustrates a flow of operations in an example method 800 for detecting methylcytosine using a modified base opposite to the methylcytosine. Method 800 may include hybridizing a first polynucleotide to a second polynucleotide (operation 802). The first polynucleotide may include methylcytosine and a plurality of cytosines. The second polynucleotide may include a modified base opposite to the methylcytosine. For example, first polynucleotide 110 may hybridize to second polynucleotide 111 in a manner such as described with reference to FIGS. 1A-1B; first polynucleotide 110 may hybridize to second polynucleotide 111 in a manner such as described with reference to FIG. 1A-1B, 2A, or 4A; first polynucleotide 310 may hybridize to second polynucleotide 330 in a manner such as described with reference to FIGS. 3A-3B; first polynucleotide 110 may hybridize to second polynucleotide 111 in a manner such as described with reference to FIGS. 3A-3B; first polynucleotide 510 may hybridize to second polynucleotide 530 in a manner such as described with reference to FIGS. 5A-5B; first polynucleotide 610 may hybridize to second polynucleotide 611 in a manner such as described with reference to FIG. 6A; or first polynucleotide 710 may hybridize to second polynucleotide 730 in a manner such as described with reference to FIG. 7A.

Method 800 also may include detecting the methylcytosine using the modified base (operation 804). For example, the modified base may include a fluorophore, and the methylcytosine may be detected using fluorescence from the fluorophore responsive to excitation light. The fluorescence may be induced using a protein. For example, the protein may couple to the methylcytosine, e.g., in a manner such as described with reference to FIG. 2A-2C or 3A-3H. For example, the coupling of the protein to the methylcytosine may dissociate the methylcytosine from the modified base while the first polynucleotide remains hybridized to the second polynucleotide. Responsive to the dissociation of the methylcytosine from the modified base, the fluorophore may fluoresce at a first intensity and a first wavelength. Responsive to the dissociation of the methylcytosine from the modified base while the first polynucleotide remains hybridized to the second polynucleotide, the fluorophore may fluoresce at a second intensity and a second wavelength. The second intensity may be different than the first intensity and/or the second wavelength is different than the first wavelength. Example modified nucleotides are provided elsewhere herein.

In other examples, the (first) protein may be coupled to a second protein, and the modified base may include a target to which the second protein selectively binds when the protein couples to the methylcytosine, e.g., in a manner such as described with reference to FIG. 4A-4D, 5A-5H, or 7A-7B. For example, the fluorophore may be coupled to the second protein. The second protein may include a second target, and the fluorophore may be coupled to a third protein that selectively binds to the second target. The second target may include an epitope, and the third protein may include an antibody. The first and second proteins may include different parts of a fusion protein. The first protein may be coupled to the second protein via a linker. Example proteins and targets are provided elsewhere herein.

### ADDITIONAL COMMENTS

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method for detecting a methylcytosine in a first polynucleotide comprising a plurality of cytosines, the method comprising:
hybridizing the first polynucleotide to a second polynucleotide,
wherein the second polynucleotide comprises a modified base opposite to the methylcytosine, wherein the modified base comprises a solvatochromatic nucleoside or a modified base comprising a target capable of coupling to a fluorophore; and
detecting the methylcytosine using the modified base.

2. The method of claim 1, wherein the modified base includes a fluorophore.

3. The method of claim 2, wherein the methylcytosine is detected using fluorescence from the fluorophore responsive to excitation light.

4. The method of claim 3, wherein the fluorescence is induced using a first protein.

5. The method of claim 4, wherein the first protein couples to the methylcytosine.

6. The method of claim 5, wherein the coupling of the first protein to the methylcytosine dissociates the methylcytosine from the modified base while the first polynucleotide remains hybridized to the second polynucleotide.

7. The method of claim 6, wherein responsive to the dissociation of the methylcytosine from the modified base, the fluorophore fluoresces at a first intensity and a first wavelength.

8. The method of claim 7, wherein responsive to the dissociation of the methylcytosine from the modified base while the first polynucleotide remains hybridized to the second polynucleotide, the fluorophore fluoresces at a second intensity and a second wavelength.

9. The method of claim 8, wherein the second intensity is different than the first intensity.

10. The method of claim 8, wherein the second wavelength is different than the first wavelength.

11. (canceled)

12. The method of claim 1, wherein the modified base comprises a modified guanine or a modified adenine.

13. (canceled)

14. The method of claim 1, further comprising coupling the methylcytosine to a first protein, wherein the first protein is coupled to a second protein, and wherein the second protein selectively binds to the first target when the first protein couples to the methylcytosine.

15. The method of claim 14, wherein a fluorophore is coupled to the second protein.

16. The method of claim 14, wherein the second protein comprises a second target, wherein the fluorophore is coupled to a third protein that selectively binds to the second target.

17. The method of claim 16, wherein the second target comprises an epitope, and wherein the third protein comprises an antibody.

18. The method of claim 14, wherein the first and second proteins comprise different parts of a fusion protein or the first protein is coupled to the second protein via a second linker.

19. (canceled)

20. The method of claim 14, wherein (a) the second protein comprises a SNAP protein and wherein the first target comprises an O-benzylguanine, or
(b) the second protein comprises a CLIP protein and wherein the first target comprises an O-benzylcytosine.

21. (canceled)

22. The method of claim 14, wherein the second protein comprises SpyTag and wherein the first target comprises SpyCatcher, or wherein the second protein comprises SpyCatcher and wherein the first target comprises SpyTag.

23. The method of claim 14, wherein the second protein comprises biotin and the first target comprises streptavidin, or wherein the second protein comprises streptavidin and the first target comprises biotin.

24. The method of claim 14, wherein the second protein comprises NTA and wherein the first target comprises His-Tag, or wherein the second protein comprises His-Tag and the first target comprises NTA.

25. The method of claim 14, wherein:
the first protein is coupled to a first half of a split fluorophore;
the second protein is coupled to a second half of a split fluorophore; and
the first half of the split fluorophore becomes coupled to the second half of the split fluorophore when the first protein becomes coupled to the methylcytosine to induce fluorescence.

26. The method of claim 14, wherein the first protein comprises a methyl binding protein (MBP) or a SET and Ring finger Associated (SRA) domain.

27. (canceled)

28. The method of claim 1, wherein the modified base is coupled to a fluorophore after the first polynucleotide is hybridized to the second polynucleotide.

29. The method of claim 1, wherein the second polynucleotide is directly coupled to a substrate or the second polynucleotide is hybridized to a third polynucleotide that is directly coupled to a substrate.

30. (canceled)

31. The method of claim 29, wherein the substrate is coupled to an oligonucleotide comprising a code identifying the first polynucleotide.

32. The method of claim 31, wherein the oligonucleotide is coupled to the substrate separately from the second polynucleotide.

33. The method of claim 32, wherein the oligonucleotide couples the second polynucleotide to the substrate.

34. The method of claim 29, wherein the substrate comprises a bead.

35. The method of claim 31, wherein detecting the methylcytosine using the modified base comprises identifying the first polynucleotide using the code.

36-70. (canceled)
